(19) United States
(12) Patent Application Publication
(10) Pub. No.: US 2006/0068494 A1
(43) Pub. Date: Mar. 30, 2006

(54) WNT4 in supporting lymphopoiesis

(76) Inventor: Claude Perreault, Montreal (CA)

Correspondence Address: FETHERSTONHAUGH - SMART & BIGGAR, 1000 DE LA GAUCHETIERE WEST, SUITE 3300, MONTREAL, QC, H3B 4W5, CA

(21) Appl. No.: 11/239,024

(22) Filed: Sep. 30, 2005

Related U.S. Application Data
(60) Provisional application No. 60/614,040, filed on Sep. 30, 2004.

(51) Int. Cl. C12N 5/06; C12N 5/08; C07K 14/47

(52) U.S. Cl. 435/325; 530/350; 435/366

(57) ABSTRACT

The T lineage differentiative potential of lymphoid progenitor cells in the lymph nodes is thwarted by the absence of Wnt signaling, resulting in blockade of the DN1→DN2 transition with accumulation of pre-DN2 cells. Wnt4 transcripts are deficient in the lymph nodes relative to the thymus. When cultured with stromal cells expressing thymus-like amounts of Wnt4 transcripts, lymphoid progenitor cells expand vigorously and generate single-positive T cells. Wnt4 can be used to promote the differentiation of lymphoid progenitors into cells such as T lymphocytes.

### REFERENCE TO RELATED APPLICATIONS

This application claims priority to U.S. provisional application 60/614,040, filed on Sep. 30, 2004, the entire content of which is herein incorporated by reference.

### FIELD OF INVENTION

The invention relates to lymphopoiesis.

### BACKGROUND OF THE INVENTION

In all animals with an adaptive immune system, the thymus is the primary lymphoid organ for T cell development. Various stages of T cell differentiation correlate with sequential migration through discrete intrathymic compartments. Congenitally athymic animals present a severe T cell lymphopenia and no other organ can compensate for defective thymic function. Progressive thymus atrophy ultimately affects all ageing subjects and can even impinge on younger subjects affected by several serious illnesses. The nature of the signals provided by thymic stromal cells that permit T cell development is unknown.

Studies have shown that a bone marrow stromal cell line ectopically expressing the Notch ligand Delta-like-1 (OP9-DL1) acquired the capacity to induce the differentiation of fetal liver derived hematopoietic progenitors and embryonic stem cells into functional T cells in vitro (1, 2). Thymus-independent T cell development can also take place in vivo by a cryptic T cell development (lymphopoietic) pathway, generating a limited number of mature T cells. In another study, T lymphopoiesis was shown to occur in lymph nodes (LN) (particularly mesenteric LN) and less in the Peyer's patches of athymic mice (3).

The cryptic T cell development pathway in the LN is amplified by signals transmitted by the leukemia inhibitory factor (LIF) receptor following prolonged exposure to mouse LIF or bovine oncostatin M (OM). About 215×10⁶ Thy1⁺CD4⁺CD8⁺ cells are present in the mesenteric LNs of 12-week-old OM-transgenic mice (4).

LNs can support in situ generation of mature single-positive (SP) T cells following i.v. injection of DN thymocytes but not of hematopoietic stem cells into athymic hosts (5).

The least mature thymocytes are termed double-negative 1 (DN1) cells and express a Lin⁻CD44⁺CD25⁻ surface phenotype. Following in vivo adoptive transfer or in vitro culture, c-Kit^{hi}IL-7Rα⁻ cells represent the DN1 subset that displays, on a per-cell basis, the most effective T precursor potential. Thymocytes subsequently go through DN2 (CD44⁺CD25⁺), DN3 (CD44⁻CD25⁺), and DN4 (CD44⁻CD25⁻) stages before giving rise to CD4⁺CD8⁺ double-positive (DP) T cells. The relation between c-Kit^{hi}IL-7Rα⁻ DN1 cells and the thymus seeding cells is a matter of controversy. According to one paradigm, the development sequence starts with bone marrow Lin⁻c-Kit^{lo}IL-7Rα⁺ common lymphoid progenitors (CLP)-1 that gives rise to a B220⁺c-Kit^{lo}IL-7Rα⁺ CLP-2 population which enters the thymus and subsequently acquires the B220⁻c-Kit^{hi}IL-7Rα⁻ phenotype. An alternative but not mutually exclusive model posits that thymic c-Kit^{hi}IL-7Rα⁻ DN1 cells (referred to as ETPs, early thymic progenitors) are not derived from CLPs but instead arise from an early bone marrow derived c-Kit^{hi}IL-7Rα⁻Flt3⁺ hematolymphoid progenitor.

The Wnt gene family consists of structurally related genes which encode secreted signaling proteins. These proteins have been implicated in oncogenesis and in several developmental processes, including regulation of cell fate and patterning during embryogenesis. The human Wnt4 gene (NCBI accession no. NM_{—}030761) is the first signaling molecule shown to influence the sex-determination cascade. It encodes a protein (NCBI accession no. NP_{—}110388; SEQ ID NO:2) which shows 98% amino acid identity to the Wnt4 protein of mouse (NCBI accession no. NP_{—}033549; SEQ ID NO:1; nucleotide NCBI accession no. NM_{—}009523) and rat. This gene and Wnt2 and Wnt7B may be associated with abnormal proliferation in breast tissue.

Wnt signaling promotes cell proliferation by increasing transcription of c-myb, c-myc, and c-fos, and decreasing that of junB. Key downstream events include induction of cyclin D2 by c-myc, and repression of two cyclin-dependent kinase inhibitor, p16^{INK4a} and p21^{Cip1/WAF1}, which are induced by junB and repressed by c-fos. Wnt signaling is complex since there are 18 Wnt proteins in mouse, and their target genes differ among various cell types (6, 7).

The transcriptional response elicited by specific Wnt proteins has not been fully characterized in immature T cells.

U.S. Pat. No. 6,159,462 (Matthews and Austin) states generally that Wnt polypeptides may be used for enhancing proliferation, differentiation or maintenance of a hematopoietic stem/progenitor cell. However, it is noted that U.S. Pat. No. 6,159,462 contains no evidence that progenitor cells can be induced to differentiate into T cells. Moreover, a paper co-authored by Matthews and Austin (25) and published at about the same time states that Wnt protein do not promote commitment of progenitors to a particular hematopoietic lineage.

### SUMMARY OF THE INVENTION

The present invention relates to the use of Wnt4 to promote the differentiation of lymphoid progenitors. More specifically, the invention relates to the development of lymphoid progenitors into T lymphocytes. Lymphoid progenitors include cells which have the phenotype of common lymphoid progenitors.

Wnt4 may be provided in the form of a purified protein, or in complex with another component such as a liposome or cell membrane to maintain its activity and/or increase its solubility. Alternatively, Wnt4 may be provided by means of cells naturally expressing Wnt4, or cells recombinantly engineered to express Wnt4 protein at a desired level. Provision of Wnt4 protein to progenitor cells to promote lymphopoiesis includes providing Wnt4 in its various forms as a cell-free protein, or in a form where Wnt4 protein is associated with the surface of expresser cells.

For example, Wnt4 protein may be provided such that it is present at a level at least equivalent to that found naturally in the thymus; this level is estimated to be at least 100 ng Wnt4 protein per mL of culture media. Wnt4 protein may be expressed on the surface of the cell, for example as a lipid-anchored protein, or it may be in secreted form.

One aspect relates to culture media comprising Wnt4 protein at a level which is at least the level of Wnt4 protein in the thymus.

Another aspect relates to a commercial package for cell or tissue culture comprising Wnt4 protein or Wnt4-expressor cells, and instructions for using the protein or expresser cells to obtain T lymphocytes from lymphoid progenitor cells. The effective level of Wnt4 for obtaining T lymphocytes is initially at least the level of Wnt4 protein in the thymus for the intended purpose of the commercial package.

Another aspect relates to a culture comprising lymphoid progenitor cells and/or T lymphocytes in media comprising Wnt4 protein at an initial level which is at least the level of Wnt4 protein in the thymus.

One way to assess suitable Wnt4 levels is to determine the level of Wnt4 protein asociated with the surface of Wnt4-expressor cells, on a per cell basis, and comparing this level with the level of Wnt4 protein on a Wnt4-expressing cell found naturally in the thymus. In this example, Wnt4-expressor cells having at least the same level of Wnt4 on their surface as do the Wnt4 producers of the thymus could be used; their numbers can be adjusted so as to allow Lymphopoiesis to occur.

Another aspect relates to an expression cassette comprising a nucleotide sequence encoding Wnt4 protein operably linked to a regulatory element such that Wnt4 expression is at a level which is at least equivalent to that found naturally in the thymus. The cassette may be used to modify a cell to produce Wnt4-expressor cells.

Another aspect relates to a cell or tissue culture comprising a Wnt4-expressor cell where the cell expresses Wnt4 protein at a level at least equivalent to that found naturally in the thymus. Wnt4 protein may be expressed on the surface of the cell, e.g. as a lipid-anchored protein, or it may be in secreted form.

Another aspect relates to a cell or tissue culture comprising a Wnt4-expressor cell and lymphoid progenitor cells in media where the Wnt4 protein, whether in soluble form or associated with the expresser cell, is at an initial level at least equivalent to that found naturally in the thymus. The culture may further comprise T lymphocytesl

Another aspect relates to a method for obtaining T lymphocytes from lymphoid progenitor cells, the method comprising the step of culturing the lymphoid progenitor cells in the presence of Wnt4 protein at a level which is at least equivalent to that found naturally in the thymus.

Another aspect relates to a method for obtaining T lymphocytes from lymphoid progenitor cells, the method comprising the step of culturing the lymphoid progenitor cells in the presence of a Wnt4-expressor cell, wherein the Wnt4-expressor cell comprises the expression cassette as described above and expresses Wnt4 protein at a level at least equivalent to that found naturally in the thymus.

Another aspect relates to a method for increasing T lymphocyte number in a subject, the method comprising the steps of:
(a) obtaining lymphoid progenitor cells from the subject;
(b) culturing the lymphoid progenitor cells in the presence of Wnt4 protein at a level which is at least equivalent to that found naturally in the thymus; and
(c) returning the T lymphocytes to the subject.

Another aspect relates to a method for increasing T lymphocyte number in a subject, the method comprising the steps of:
(a) obtaining lymphoid progenitor cells from the subject;
(b) culturing the lymphoid progenitor cells in the presence of a Wnt4-expressor cell to obtain T lymphocytes, wherein the Wnt4-expressor cell comprises the expression cassette described above and expresses Wnt4 protein at a level which is at least equivalent to that found naturally in the thymus; and
(c) returning the T lymphocytes to the subject.

Another aspect relates to a method for increasing T lymphocyte number in a subject, the method comprising the step of delivering Wnt4-expressor cells to the subject; wherein the Wnt4-expressor cells are delivered to an extrathymic lymphoid tissue containing lymphoid progenitor cells; and wherein the Wnt4-expressor cell comprises the expression cassette as described above and expresses Wnt4 protein at a level which is at least equivalent to that found naturally in the thymus.

Another aspect relates to a method for increasing T lymphocyte number in a subject, the method comprising the step of delivering DNA comprising the expression cassette as described above to the subject; wherein the DNA is delivered to an extrathymic lymphoid tissue containing lymphoid progenitor cells; and wherein Wnt4 protein is expressed from the expression cassette at a level which is at least equivalent to that found naturally in the thymus.

### BRIEF DESCRIPTION OF THE DRAWINGS

FIG. 1. Surface phenotype of Lin⁻ cell subsets in the wt thymus, wt LN, and OM⁺ LN.

(a) To estimate the proportion of cells with DN1-DN4 phenotype, lymphoid cells were stained for CD25, CD44, and lineage markers (CD3ε, CD8α, CD8β, CD11b, CD45R/B220, Ly6C, Ly6G, NK1.1, TER-119, TCRβ, and TCRγ). Numbers in the various quadrants correspond to percentage of Lin⁻ cells stained with CD25 and CD44. One representative experiments out of three. (b) Number of cells with DN1-DN4 phenotype in the three lymphoid organs (mean±SD; n=3). (c) Expression of c-Kit on DN1 phenotype cells (Lin⁻CD44⁺CD25⁻) One representative experiments out of three. (d) Number of c-Kit⁻, c-Kit^{lo}, and c-Kit^{hi} DN1 phenotype cells per 10⁶ lymphoid cells. Gated Lin⁻CD44⁺CD25⁻ cells were stained for c-Kit (mean±SD; n=3). No DN1 phenotype c-Kit^{hi} cells were detected in the wt and OM⁺ LNs (*). (e) Thymic DN1 subsets defined according to c-Kit levels were further characterized for expression of IL-7Rα, HSA (CD24), and Sca-1. Negative control staining is shown as dotted lines in IL-7Rα and Sca-1 panels. MFI is shown for IL-7Rα staining.

FIG. 2. T cell commitment of lymphoid progenitors in the LNs.

RT-PCR analysis of DN1 (a), pre-DN2 (b), and DN4 (e) cells sorted from wt thymus, wt LN and OM⁺ LN. One step RT-PCR was done on the same mRNA samples for transcripts of interest and Hprt. (c) c-Kit expression on DN2 and DN3 subsets from wt thymus (dotted line) and OM⁺ LN (solid line). Intracellular TCRβ (icTCRβ) chain expression in DN3 (d) and DN4 (f) subsets. Dotted lines represent staining with isotype control antibodies. (g) 10⁵ sorted DN4 cells (Lin⁻CD8⁻CD44⁻CD25⁻) harvested from wt LNs were co-cultured on OP-9-DL1 cells and analyzed for T cell development after 7 days of in vitro culture. Numbers indicate cell population percentages.

FIG. 3. Survival and proliferation of DN cells are impaired in wt LN.

(a) Analysis of cell cycle status of DN phenotype cells. Forty min after injection of 1 mg BrdU i.p., mice were sacrificed, and cells were stained with 7AAD and antibodies against BrdU, CD25, CD44 and lineage markers. Numbers correspond to the percentages of cells in the G_{1/0}, S, and G₂+M phase of the cell cycle. One representative experiment out of three. (b) Lin⁻ CD44⁺c-Kit subsets in cycling thymocytes. Mice received two injections (1 mg each) of BrdU at 2 h interval. 24 h later, prepared cells were stained with antibodies against BrdU, CD44, c-Kit and lineage markers (which included CD25). One representative experiment out of three. (c) Percentage of AnnexinV⁺ cells in wt thymus, wt LN, and OM⁺ LN.

FIG. 4. Quantitative real time PCR analysis on thymic and lymph node stromas and OP-9 DL-1 cells.

(a) mRNA expression profile of selected genes in the stroma of wt thymus and LN, OM⁺ LN, and OP-9 DL-1 cells. (b) levels of Wnt4 transcripts in the stromal and lymphoid fraction of lymphoid organs. mRNA values where normalized according to Hprt and thymic stroma mRNA levels were set as 1. Data are mean±SD from three or four independent experiments (* indicates no detectable mRNA after 50 amplification cycles). Differences between groups were evaluated with Student's t test. Levels of statistical significance for comparison of wt thymus vs. wt LN are ¥ P<0.04, § P<0.005, and ‡ P<0.0001. † Levels of DL-1 transcript\grave{s} for OP-9 DL1 cells (715±55) are not shown on the graph.

FIG. 5. Wnt and LIF/OM signalling pathways in DN cells.

Real-time RT-PCR assays and FACS analysis measuring gene and protein expression in DN1 (a, b), pre-DN2 (c, d), and DN4 (e, f) cells sorted from lymphoid organs. The mRNA levels of the wt thymus for DN1 (a) and DN4 (e) cells, and of wt LN for pre-DN2 (c) cells were set as 1. Hprt mRNA levels were used to normalize cDNA content among subpopulations. Data are mean±SD from three independent experiments. Differences between groups were evaluated with Student's t test. Levels of statistical significance for comparison of wt thymus vs. wt LN are *P<0.02 and **P<0.003, and for comparison of wt LN vs. OM⁺ LN, †P<0.04, ††P<0.008, and †††p<2×10⁻⁶. For protein expression, intracellular (bcl-2 and phospho-Stat3 (P-Stat3)) and surface staining (CD44) were done (b, d and f) on wt thymus (slim black line), wt LN (thick black line) and OM⁺ LN (close dotted line) DN cells. Secondary antibody was used as a negative control for P-Stat3 staining (broad dotted line).

FIG. 6. c-Kit^{lo}IL-7Rα⁺ and c-Kit^{hi}IL-7Rα⁻ progenitors display different differentiation potential when grown on OP9 and OP9-DL1 cells.

The following subsets of Lin⁻CD44⁺CD25⁻ DN1 cells were sorted: c-Kit^{lo}Sca-1⁺ cells from the thymus, wt LN and OM⁺ LN, and c-Kit^{hi}Sca-1⁺ cells from the thymus. These DN1 cell populations were plated on confluent monolayer of (a) OP-9-GFP cells or (b) OP-9-DL1 cells, and analyzed by flow cytometry at the indicated time points. Fold expansion was measured by dividing the number of cells harvested by the number of cells initially plated.

FIG. 7. LN c-Kit^{lo}IL-7Rα⁺ progenitors can complete T cell development when grown on OP9-DL1-Wnt4 cells.

(a) 4×10³ sorted DN1 cells (Lin⁻CD44⁺CD25⁻Sca1⁺c-kit^{lo}) from wt LN were plated 6 well tissue culture plates containing a confluent monolayer of OP-9-DL1 cells expressing or not Wnt4, and analyzed by flow cytometry after 12 days in culture. (b) 10⁵ sorted DN4 cells (Lin⁻CD44⁻CD25⁻) harvested from wt LN were co-cultured on OP-9-DL1 cells overexpressing or not Wnt4, and analyzed on day 7. Numbers indicate cell population percentages.

FIG. 8. Relative transcript levels of OP9-DL1 cells, OP9-DL1-Wnt4 cells, and stromal cells from wt LN, OM+ LN.

Real-time PCR is performed simultaneously on both the reference sample and the experimental samples containing RNA extracted from OP9-DL1 cells, OP9-DL1-Wnt4 cells, stromal cells from wt LN, and stromal cells from OM+ LN. A relative value for target gene expression in each sample is extrapolated from the standard curve generated from the reference sample. For each sample, the ratio of target gene/HPRT expression is calculated. The reference sample ratio (thymic stroma) is then arbitrarily set at 1 and each sample ratio values are then transformed proportionately.

### DETAILED DESCRIPTION OF EMBODIMENTS

The invention stems from, but is not limited to, the discovery that the T lineage differentiative potential of Lin⁻c-Kit^{lo}IL-7Rα⁺ cells in the LNs is thwarted by the absence of Wnt signaling, specifically Wnt4 signaling, resulting in blockade of the DN1→DN2 transition with accumulation of pre-DN2 cells. Wnt4 transcripts, a ligand produced by stromal cells that regulate early steps of T cell development, were deficient in the LN relative to the thymus. When cultured with stromal cells expressing thymus-like amounts of Wnt4 transcripts, Lin⁻c-Kit^{lo}Sca-1⁺IL-7Rα⁺ lymphoid progenitor cells from the LN expand vigorously and generate single-positive T cells.

It is noted that c-Kit^{lo}Sca-1⁺IL-7Rα⁺ populations, whether they were from the thymus or LNs, had the same behavior when cultured in vitro. Their phenotype and their ability to generate T and B cells suggest that they are closely related to bone marrow common lymphoid progenitors (CLP).

A recent study showed that CLP-derived T cells can protect against lethal murine cytomegalovirus infection (8). Our demonstration that Wnt4 can amplify T cells from CLP-phenotype cells is thus relevant for treatment of subjects with T cell lymphopenia.

The Wnt pathway is highly conserved and there is a 98% amino acid identity between mouse and human Wnt4. We expect an appropriate amount of Wnt4 to be useful for developing ex vivo cultures to generate therapeutically useful numbers of T lymphocytes from blood or bone marrow lymphoid progenitors.

For ease of reference, the following abbreviations and designations are used throughout:
LN lymph node
DL1 Notch ligand Delta-like-1
LIF leukemia inhibitory factor
OM Oncostatin M
DN double negative; with reference to thymocytes, DN cells are Th-1⁺CD4⁻CD8⁻. DN1 cells express a Lin⁻CD44⁺CD25⁻ surface phenotype; DN2 are CD44⁺CD25⁺; DN3 are CD44⁻CD25⁺; and DN4 are CD44⁻CD25⁻.
DP double positive (Th-1⁺CD4⁺CD8⁺)
SP single positive (Th-1⁺CD4⁺CD8⁻ or Th-1⁺CD4⁻CD8⁺)
CLP common lymphoid progenitor
wt wild type
TCR T cell receptor
ETP early thymic progenitor
BrdU 5-Bromo-2deoxyuridine
HSC hematopoietic stem cell
   Wnt4 Polypeptide, Nucleic Acid, Expression

Wnt-4 polypeptides or proteins as used in the present invention include any of the Wnt4 homologues known in the art and variants thereof. Such include the zebrafish, frog, chicken, mouse, rat and human sequences identified by NCBI accession numbers P47793, A49146, NP_{—}990114, NP_{—}033549, NP_{—}445854 and NP_{—}110388 respectively. The human sequence is preferred.

The Wnt4 sequences contemplated also include variants which have at least 90% identity, 98% identity and 99% identity to the human Wnt4 amino acid sequence over its entire length. Preferably the Wnt-4 variants exhibit at least one biological activity of the native (wt) Wnt-4, in particular Wnt4 activities in the differentiation of lymphoid progenitors.

Notably, among the Wnt4 homologues, the human Wnt4 amino acid sequence is 98% identical with the mouse and the rat sequences, using the BLAST sequence alignment software set under standard parameters. The human Wnt4 amino acid sequence is 82%, 83% and 86% identical with the zebrafish, frog and chicken sequences respectively, using the same alignment parameters.

The Wnt4 polypeptides may be in the form of the “mature” protein, a biologically active fragment capable of inducing lymphopoiesis, or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Wnt4 polypeptides may be in a form soluble in culture media, or may be in a form associated with the sruface of an expresser cell. Wnt4 may be effective as a monomer or as a multimer. In the present context, Wnt4 polypeptides, when used in an effective amount, promote Lymphopoiesis.

Wnt4 is a member of the Wnt family of proteins and so may share certain characteristics with other members of this family. In their naturally occurring forms, human WNT proteins are generally insoluble and have cysteine residues the spacing of which is highly conserved. Thus Wnt protein folding and structure may depend on the formation of multiple intramolecular disulfide bonds. The carboxy-terminal region of Wnt4 protein may be important in determining the Wnt4-specific responses, while the amino-terminal region may mediate interactions with Wnt receptors but requires the carboxyl terminus to activate these receptors.

Natural Wnt proteins are expressed with an amino-terminal signal sequence and are present in the secretory pathway, indicating that they are secreted proteins. They associate with glycosaminoglycans in the extracellular matrix and are bound tightly to the cell surface. Although Wnts are found in tight association with the plasma membrane, it is reported that active Wnt may be obtained from the medium of cultured cells.

Reception and transduction of Wnt signals involves binding of Wnt proteins to members of two distinct families of cell surface receptors, members of the Frizzled gene family and members of the LDL-receptor-related protein (LRP) family.

In some embodiments, Wnt4 polypeptide is used in a form that is purified, isolated or substantially pure. Wnt4 is “substantially pure” when it is separated from the components that naturally accompany it. Typically, a compound is substantially pure when it is at least 60%, more generally 75% or over 90%, by weight, of the total material in a sample. Thus, for example, a polypeptide that is chemically synthesised or produced by recombinant technology will generally be substantially free from its naturally associated components. A nucleic acid molecule is substantially pure when it is not immediately contiguous with (i.e., covalently linked to) the coding sequences with which it is normally contiguous in the naturally occurring genome of the organism from which the DNA of the invention is derived. A substantially pure compound can be obtained, for example, by extraction from a natural source; by expression of a recombinant nucleic acid molecule encoding a polypeptide compound; or by chemical synthesis. Purity can be measured using any appropriate method such as column chromatography, gel electrophoresis, HPLC, etc.

Wnt4 polypeptide may be produced as a fusion protein where the Wnt4 sequence is fused in-frame to a heterologous polypeptide such as the commercially available His-tag. Fusion to the C-terminus of Wnt4 is preferred. An amino acid cleavage site is optionally placed between the Wnt4 sequence and the heterologous polypeptide.

A simple way to obtain such a fusion polypeptide is by translation of an in-frame fusion of the polynucleotide sequences, i.e., a hybrid gene. The hybrid gene encoding the fusion polypeptide is inserted into an expression vector which is used to transform or transfect a host cell. Alternatively, the Wnt4 polynucleotide sequence is inserted into an expression vector in which the polynucleotide encoding the heterologous polypeptide is already present. Such vectors and instructions for their use are commercially available, e.g. the pMal-c2 or pMal-p2 system from New England Biolabs, in which the heterologous polypeptide is a maltose binding protein, the glutathione-S-transferase system of Pharmacia, or the His-Tag system available from Novagen. These and other expression systems provide convenient means for isolating and purifying Wnt4 protein.

Amino acids that may be used to link Wnt4 to the heterologous polypeptide include aspartic acid-proline, asparagine-glycine, methionine, cysteine, lysine-proline, arginine-proline, isoleucine-glutamic acid-glycine-arginine, and the like. Cleavage may be effected by exposure to the appropriate chemical reagent or cleaving enzyme.

It should be recognized that cleavage may not be necessary for Wnt4 protein to be useful in the present application. A cleavage site could be incorporated, or absent.

Wnt4 protein may thus be produced by transforming a compatible host with a vector suitable for expressing a fusion polypeptide containing Wnt4, culturing the host, isolating the fusion polypeptide by selective binding to an affinity matrix such as a carrier linked to an antibody specific for the heterologous polypeptide, and cleaving off the Wnt4 protein either directly from the carrier-bound fusion polypeptide or after desorption from the carrier.

A necessary condition to permit such cleavage of the produced polypeptide is that it contains a unique cleavage site which may be recognized and cleaved by suitable means. Such a cleavage site may be a unique amino-acid sequence recognizable by chemical or enzymatic means and located between the Wnt4 polypeptide and the heterologous polypeptide. Such a specific amino acid sequence should not occur within the Wnt4 portion.

Examples of enzymatic agents include proteases, such as collagenase, which in some cases recognizes the amino acid sequence NH₂-Pro-X-Gly-Pro-COOH, wherein X is an arbitrary amino acid residue, e.g. leucine; chymosin (rennin), which cleaves the Met-Phe bond; kallikrein B, which cleaves on the carboxyl side of Arg in X-Phe-Arg-Y; enterokinase, which recognizes the sequence X-(Asp)ₙ-Lys-Y, wherein n=2-4, and cleaves it on the carboxyl side of Lys; thrombin which cleaves at specific arginyl bonds. Examples of chemical agents include cyanogen bromide (CNBr), which cleaves after Met; hydroxylamine, which cleaves the Asn-Z bond, wherein Z may be Gly, Leu or Ala; formic acid, which in high concentration (about 70%) specifically cleaves Asp-Pro. Thus, if the desired portion does not contain any methionine sequences, the cleavage site may be a methionine group which can be selectively cleaved by cyanogen bromide. Chemical cleaving agents may be preferred in certain cases because protease recognition sequences may be sterically hindered in the produced polypeptide.

The techniques for introducing DNA sequences coding for such amino acid cleavage sites into the DNA sequence coding for the polypeptide are well-known in the art.

As mentioned above, cleavage may be effected either with the fusion polypeptide bound to the affinity matrix or after desorption therefrom. A batch-wise procedure may be carried out as follows. The carrier having the fusion polypeptide bound thereto, e.g. IgG-Sepharose where the IgG is specific against the heterologous polypeptide, is washed with a suitable medium and then incubated with the cleaving agent, such as protease or cyanogen bromide. After removal of the carrier material having the heterologous polypeptide bound thereto, a solution containing the cleaved desired polypeptide and the cleavage agent is obtained, from which the former may be isolated and optionally further purified by techniques known in the art such as gel filtration, ion-exchange etc.

Where the fusion polypeptide comprises a protease recognition site, the cleavage procedure may be performed in the following way. The affinity matrix-bound fusion polypeptide is washed with a suitable medium, and then eluted with an appropriate agent which is as gentle as necessary to preserve the Wnt4 activity. Such an agent may be a pH-lowering agent such as a glycine buffer. The eluate containing the pure fusion polypeptide is then passed through a second column comprising the immobilized protease, e.g. collagenase when the cleavage site is a collagenase susceptible sequence. When passing therethrough the fusion polypeptide is cleaved into the desired Wnt4 protein and the heterologous polypeptide. The resulting solution is then passed through the same affinity matrix, or a different affinity matrix, to adsorb the heterologous polypeptide portion of the solution.

Fragments of the Wnt-4 polypeptides may also be used. A fragment is a polypeptide having an amino acid sequence that is the same as part, but not all, of the Wnt4 amino acid sequence. Representative fragments include, for example, fragments from about amino acid number 1-20, 21-40, 41-60, 61-80, 81-100, and 101 to the end of the Wnt-4 sequence. In this context “about” includes the particularly recited ranges larger or smaller by several, 5, 4, 3, 2 or 1 amino acid at either extreme or at both extremes. It is contemplated that in some embodiments, the truncations, variants and fragments of Wnt4 may retain amino acid residues 23-57 in the mature human Wnt4 protein or the corresponding region in Wnt4 homologs, to preserve Wnt4-specific activity.

Preferred fragments include, for example, truncation polypeptides having the amino acid sequence of Wnt-4 polypeptides, except for deletion of a continuous series of residues that includes the amino terminus, or a continuous series of residues that includes the carboxyl terminus or deletion of two continuous series of residues, one including the amino terminus and one including the carboxyl terminus. Also preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate Wnt-4 activity, including those with a similar activity or an improved activity, or with a decreased undesirable activity.

Preferred Wnt4 truncations and variants include those having increased solubility. For example, the Wnt4 sequence may be modified to remove the lipid acylation sequence responsible for palmitoylation of the protein. Palmitoylation of Wnt4 is believed to occur at the first conserved cysteine, corresponding to Cys79 of human Wnt4. Preferred Wnt4 truncations include those which are more soluble than the full-length sequence, and which retain the ability to induce lymphopoiesis. Where the more soluble variants and truncations of Wnt4 are used, it is believed that the level required for inducing T cell development may be higher than that found naturally in the thymus. It is contemplated that a level at least 1.5 times, 2 times, 3 times, 4 times or higher than the level of Wnt4 in the thymus may be required. This higher level may be required at the initial stages so that lymphopoiesis may be induced. Subsequently, the Wnt4 level may be lower for maintaining lymphopoiesis and the production of T cells.

Preferred variants are those that vary from the native Wnt4 sequence by conservative amino acid substitutions; i.e., those that substitute a residue with another of like characteristics. Typical substitutions include those among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

The Wnt-4 polypeptides may be prepared in any suitable manner as known in the art. Such polypeptides include isolated naturally occurring polypeptides, recombinantly produced polypeptides, synthetically produced polypeptides, or polypeptides produced by a combination of these methods. Means for preparing such polypeptides are well understood in the art.

Certain aspect of the invention uses Wnt-4 polynucleotides. These include isolated polynucleotides which encode the Wnt-4 polypeptides, variants and fragments defined above.

Methods to determine identity and similarity are codified in publicly available computer programs. Preferred computer program methods to determine identity and similarity between two sequences include, but are not limited to, the GCG program package, BLASTP, BLASTN, and FASTA. The BLAST X program is publicly available from NCBI and other sources. The well known Smith Waterman algorithm may also be used to determine identity.

Preferred parameters for polypeptide sequence comparison include the following:
Algorithm: Needleman and Wunsch, J. Mol. Biol. 48: 443-453 (1970);
Comparison matrix: BLOSSUM62 from Hentikoff and Hentikoff, Proc. Natl. Acad. Sci. USA. 89:10915-10919 (1992);
Gap Penalty: 12
Gap Length Penalty: 4

A program useful with these parameters is publicly available as the “gap” program from Genetics Computer Group, Madison, Wis. The aforementioned parameters are the default parameters for amino acid sequence comparisons (along with no penalty for end gaps).

Wnt4 Levels

The present invention relates to use of Wnt4 polypeptide at a level at least comparable to that of the thymus for inducing lymphopoiesis in lymphoid progenitor cells. The present invention also relates to expression cassettes and vectors which comprise the Wnt4 sequence, and host cells which are genetically engineered with the expression cassettes to express/produce Wnt4 by recombinant techniques. The invention also includes use of Wnt4-expressor cells as a means of expressing Wnt4 polypeptide at a level at least comparable to that of the thymus, in the lymphoid progenitor cell culture. This level may be required at the initial stages so that lymphopoiesis may be induced. Subsequently, the Wnt4 level may be lower for maintaining lymphopoiesis and the production of T cells.

Where Wnt4 polypeptide is stated to be at a level at least comparable to that of the thymus, this is intended to mean that Wnt4 polypeptide is present at a level of biological activity equivalent to the thymus environment. This level in vitro, i.e. in culture media or in a cell culture, is estimated to be about at least 100 ng Wnt4 protein per mL media or culture. The level may be somewhat lower (e.g. 80-90 ng/mL), or higher depending on the specific activity of the Wnt4 preparation or source. The useful higher level may be 120 ng/mL, 150 ng/mL, 200 ng/mL, 300 ng/mL up to 500 ng/mL. Wnt4 protein levels may be determined by methods known in the art such as by use of anti-Wnt4 antibodies, or by use of antibodies against a fusion tag, e.g. His tag, to which recombinant Wnt4 is fused.

Antibodies against mouse Wnt4 are commercially available (R&D Systems, Inc. 614 McKinley Place NE Minneapolis, Minn. 55413). These include goat polyclonal antibodies against mouse a Wnt4 fusion protein (residues 37-76 and 222-295 joined by a linker, with 6 histidine residues at the C-terminus), and monoclonal antibodies against mouse Wnt4 residues 37-76. A Wnt4-specific antibody made against the peptide sequence 23-36 of human Wnt4 (SEQ ID NO:2), has also recently become commercially available (Imgenex 11175 Flintkote Ave., Suite E, San Diego, Calif. 92121). It is understood that conditions in which the antibodies are used be optimized by routine assays.

As an alternative means of determining the level of Wnt4 which is at least comparable to that of the thymus, functional assays may be carried out. Wnt4 functional assays are known in the art and include those based on Wnt4's involvement in canonical Wnt/beta-catenin activation pathway and the non-canonical Wnt/Calcium activation pathway.

In an assay based on the Wnt/beta-catenin activation pathway, Wnt4 level is determined indirectly via beta-catenin-mediated TCF/LEF transcriptional activity using the TOPFLASH reporter gene which contains TCF/LEF binding sites. The assay is described in Xiao et al. (26).

In an assay based on the Wnt/Calcium activation pathway, this pathway involves activation of a heterotrimeric G protein, an increase in intracellular calcium, and activation of calcium/calmodulin-regulated kinase II and PKC. Assays to monitor activation of the Wnt/Calcium activation pathway are described in Sheldahl et al. (27).

Where Wnt4 is stated to be expressed at a level at least comparable to that of the thymus, this is intended to mean that Wnt4 expression is comparable to Wnt4 expression in cells of the thymus. The level of Wnt4 expression may be determined by assessing Wnt4 transcript levels in the Wnt4-expressor cells and thymus cells, using methods known in the art. Such methods include Northern hybridizations and quantitative PCR. A determination of relative amounts, e.g. transcript levels in expresser cells compared to that in thymus cells, may be made by normalizing against the expression levels of a house-keeping gene, such as HPRT. It is contemplated that a transcript level at least equal to, or 1.5 times, 2 times, 3 times, 4 times or higher than the level of Wnt4 in the thymus may be required for inducing T cell development from stem/progenitor cells in vitro or outside the thymus.

One skilled in the art would understand that not all vectors and expression control sequences and hosts would be expected to express equally well the polynucleotides of this invention. With the guidelines described below, however, a selection of vectors, expression control sequences and hosts may be made without undue experimentation and without departing from the scope of this invention.

In selecting a vector, the host must be chosen that is compatible with the vector which is to exist and possibly replicate in it. Considerations are made with respect to the vector copy number, the ability to control the copy number, expression of other proteins such as antibiotic resistance.

In selecting an expression control sequence, a number of variables are considered. Among the important variable are the relative strength of the sequence (e.g. the ability to drive expression under various conditions), the ability to control the sequence's function, compatibility between the polynucleotide to be expressed and the control sequence (e.g. secondary structures are considered to avoid hairpin structures which prevent efficient transcription).

In selecting the host, unicellular hosts are selected which are compatible with the selected vector, tolerant of any possible toxic effects of the expressed product, able to secrete the expressed product efficiently if such is desired, to be able to express the product in the desired conformation, to be easily scaled up, and to which ease of purification of the final product.

The expression cassette is typically part of an expression vector, which is selected for its ability to replicate in the chosen expression system. Suitable expression vectors can be purchased from various commercial sources.

The choice of the expression cassette depends on the host system selected as well as the features desired for the expressed polypeptide. Typically, an expression cassette includes a promoter that is functional in the selected host system and can be constitutive or inducible; a ribosome binding site; a start codon (ATG) if necessary; a region encoding a signal peptide, e.g., a lipidation signal peptide; a DNA molecule of the invention; a stop codon; and optionally a 3′ terminal region (translation and/or transcription terminator). The signal peptide encoding region is adjacent to the polynucleotide of the invention and placed in proper reading frame. The signal peptide-encoding region is homologous or heterologous to the DNA molecule encoding the mature polypeptide and is compatible with the secretion apparatus of the host used for expression. The open reading frame constituted by the DNA molecule of the invention, solely or together with the signal peptide, is placed under the control of the promoter so that transcription and translation occur in the host system.

Promoters and signal peptide encoding regions are widely known and available to those skilled in the art. The promoters may be either naturally occurring promoters or hybrid promoters, which combine elements of more than one promoter. In addition, the expression vector may comprise additional elements. For example, the expression vector may have two replication systems, thus allowing it to be maintained in two organisms, for example in mammalian or insect cells for expression and in a prokaryotic host for cloning and amplification. For integrative expression vectors, the expression vector contains at least one sequence homologous to the host cell genome, and preferably two homologous sequences which flank the expression construct. Constructs for integrative vectors are well known in the art.

The Wnt4 proteins used in the present invention may be produced by culturing a host cell transformed with an expression cassette containing a nucleic acid encoding a Wnt4 protein under the appropriate conditions to induce or cause expression of the protein.

Appropriate host cells include yeast, bacteria, archebacteria, fungi, and insect and animal cells, including mammalian cells. These include Drosophila melangaster cells, Saccharomyces cerevisiae and other yeasts, E. coli, Bacillus subtilis, SF9 cells, C129 cells, 293 cells, Neurospora, BHK, CHO, COS, and HeLa cells, fibroblasts, Schwanoma cell lines, immortalized mammalian myeloid and lymphoid cell lines such as Jurkat and BJAB cells. Of particular interest are mammalian stromal cells such as OP9 and OP9-DL1 cells (available upon request from Dr. J. C. Zuniga-Pflucker, University of Toronto), CRL-2496, CRL-2647 and CRL-11882 cells (available from the American Type Culture Collection).

In certain embodiment, Wnt4 proteins are expressed in mammalian cells. Such Wnt4 expressors may be used as a means for producing Wnt4 polypeptide. The Wnt4-expressing cells may also be used in co-culture with the progenitor/stem cells. Co-culture of Wnt4-expressor cells with the progenitor/stem cells may be advantageous where Wnt4 protein is associated with the surface of the expressor cells.

Mammalian expression systems are known in the art, and include retroviral systems. Of particular use are expression systems and promoters that allow expression of Wnt4 protein to a level at least comparable to that in the thymus and support differentiation of hemapoietic stem cells and lymphoid progenitor cells. Promoters contemplated for use to achieve Wnt4 expression levels comparable to the thymus include the CMV promoter and the PGK promoter.

A promoter will have a transcription initiating region, which is usually placed proximal to the 5′ end of the coding sequence, and a TATA box, using a located 25-30 base pairs upstream of the transcription initiation site. A mammalian promoter will also contain an upstream promoter element (enhancer element), typically located within 100 to 200 base pairs upstream of the TATA box. An upstream promoter element determines the rate at which transcription is initiated and can act in either orientation.

Of particular use are the promoters from mammalian viral genes, since the viral genes are often highly expressed and have a broad host range.

Promoters may be from the genomes of viruses such as polyoma virus, fowlpox virus, adenovirus (such as Adenovirus 2), bovine papilloma virus, avian sarcoma virus, cytomegalovirus, retrovirus, hepatitis-B virus and Simian Virus 40 (SV40). Examples include the SV40 early promoter, mouse mammary tumor virus LTR promoter (MMTV), adenovirus major late promoter, herpes simplex virus promoter, the thymidine kinase promoter from herpes simplex virus, the Rous sarcoma virus long terminal repeat (RSV-LTR) promoter, and the cytomegalovirus (CMV) promoter.

Promoters may also be heterologous mammalian promoters, e.g., the PGK promoter, the actin promoter or an immunoglobulin promoter, and heat-shock promoters. It is understood that such promoters should be compatible with the host cell expression systems.

Expression of a DNA encoding the Wnt4 polypeptide as described herein is often increased by inserting an enhancer sequence into the vector. Enhancers are cis-acting elements of DNA, usually about from 10 to 300 bp, that act on a promoter to increase its transcription. Enhancers are relatively orientation and position independent, having been found 5′ and 3′ of the transcription unit, within an intron, as well as within the coding sequence itself. Enhancer sequences from mammalian genes include globin, elastase, albumin, alpha-fetoprotein, and insulin. Enhancers from eukaryotic cell viruses include the SV40 enhancer, the cytomegalovirus early promoter enhancer, the polyoma enhancer, and adenovirus enhancers.

Cell Culture

The present invention provides a cell culture media which supports the differentiation and/or growth of hemapoietic stem cells and lymphoid progenitor cells into T cells. Preferably, the cells are human cells.

The media for use in the cell culture to support the differentiation and/or growth of hemapoietic stem cells and lymphoid progenitor cells into T cells contains Wnt4 protein. Preferably the level of Wnt4 in the culture medium is at least the level of Wnt4 protein as found in the thymus of an immuno-competent subject. The level of Wnt4 protein in the culture medium is preferably at least 100 ng/mL culture medium. The level may be somewhat lower (e.g. 80-90 ng/mL), or higher depending on the specific activity of the Wnt4 preparation or source. The useful higher level may be 120 ng/mL, 150 ng/mL, 200 ng/mL, 300 ng/mL up to 500 ng/mL.

Wnt4 protein in the culture media may be supplied exogenously, in the form of Wnt4 polypeptide, or by way of cells which express the Wnt4 protein in an effective level, and which are compatible with the progenitor cell culture. Such Wnt4-expressor cells may be cells modified to express Wnt4 recombinantly. The Wnt4-expressor cells may also be stromal cells. These are large, spread-out cells that provide a bed for hematopoietic cells. The stromal cells may also be modified by having a Wnt4-encoding sequence introduced recombinantly into them.

The cells may express Wnt4 protein into the culture media, or may express Wnt4 at the cell surface. Wnt4 may be expressed at a level at least comparable to that of the thymus, or at an effective level to permit differentiation and/or growth of hemapoietic stem cells and lymphoid progenitor cells into T cells. In a culture media, the initial level of Wnt4, i.e. the level of Wnt4 at the start of the culture for producing T cells, may be at a level at least comparable to that of the thymus, or at an equivalent effective level. Wnt4 may be in soluble form or in a form associated with the cell surface of the expressor cell.

Stromal cells are derived from mesenchymal stem cells, are not of hematopoietic origin, express class 1 histo-compatibility antigens, but lack the hematopoietic cell surface marker CD45. Stromal cells may include such cells as endothelial cells, reticular cells, fat cells and professional antigen presenting cells such as dendritic cells. The stromal cells may be isolated from many different sources such as e.g., adult and fetal bone marrow, spleen, thymus, peripheral blood, liver, umbilical cord, para-aortic splanchnopleura, aorta, gonads and mesonephros (AGM), lymph node, and other types of stromal cells, or derived from stem cells such as e.g., bone marrow stem cells, peripheral blood cells, peripheral stem cells, embryonic stem cells, umbilical cord cells, umbilical blood stem cells, embryonic stem cells, other types of stem cells, or any combination of these cells. An example of a stromal cell line is OP9-DL1. The stromal cell line may be transfected transiently or integratively with a Wnt4 expression cassette so that Wnt4 protein is expressed, as a soluble protein or as a cell-surface protein, at a level in the culture media at least comparable to the Wnt4 level in the thymus.

Expression of Wnt4 at an effective level may be achieved by having the Wnt4 sequence operably linked to a high expression promoter such as the CMV promoter, and other regulatory elements such as enhancers.

Exogenously added Wnt4 polypeptide may be supplied in a form which keeps the Wnt4 protein functional in the culture media, for example by having Wnt4 protein in complex with a liposome or with the cell membrane.

The presence of Wnt 4 at a level at least comparable to the Wnt4 level in the thymus supports the maturation or differentiation of the stem and progenitor cells into T lymphocytes. The increased content of T cells is observable by various means known in the art, such as techniques which monitor the T cells by staining for their cell surface markers. The cell culture containing the Wnt4-promoted T cells may contain 1.1-fold to 100-fold the number of T cells originally in the culture. Alternatively, the cell culture containing the Wnt4-promoted T cells may have an increased proportion of T cells compared to the proportion originally in the culture. Relative to a control culture where the cells are grown without Wnt4 or at low levels of Wnt4, the proportion of T cell present may be 2-fold, 4-fold, 5-fold, 10-fold, up to the point where the culture consists substantially of T cells instead of the original stem cells and progenitor cells.

Included in the culture media may be interleukin-7 (IL-7), c-kit ligand and Notch ligand such as that expressed by the stromal cell line OP9-DL1. Cytokines or other molecules which may also be used in the media include for example, interleukin-2, interleukin-12, slt-3L, CD40L, interleukin-4, interleukin-10, interleukin-6, BCF-1, and stem cell factor.

Lymphoid Progenitors

A hematopoietic stem cell or lymphoid progenitor cell is one which is able to differentiate to form a more committed or mature blood cell type; in the context of the present invention specifically a lymphocyte, more specifically a T lymphocyte.

Lymphoid progenitor cells are those hematopoietic precursor cells which are able to differentiate to form lymphocytes (B-cells or T-cells). Lymphopoiesis is the formation of lymphocytes.

Hemapoietic stem cells include bone marrow stem cells, peripheral blood stem cells, embryonic stem cells, stem cells from umbilical cord and stem cells from other sources. These stem cells may be obtained from bone marrow, blood or cord blood.

Lymphoid progenitor cells are a subset of hemapoietic stem/progenitor cells and are precursors to lymphocytes. They include cells from the lymphoid organs which are precursors to T cells and B cells. The progenitor cells may be obtained from lymphoid organs which include the thymus (primary immune organ), lymph nodes, lymphatic vessels, spleen, unencapsulated lymphoid tissue including the lingual, palatine and pharyngeal tonsils, the small intestinal Peyer's patches, the appendix, and the lymphoid mucosa (secondary immune organs).

Functionally, hematopoietic stem cells are capable of prolonged self-renewal and differentiation into all the hematopoietic cell lineages. Thus, hematopoietic stem cells, when localized to the appropriate microenvironment, can completely and durably reconstitute the hematopoietic and lymphoid compartments.

Multi-lineage stem and progenitor cells can also be identified phenotypically by cell surface markers. A number of phenotypic markers, singly and in combination, have been described to identify the pluripotent hematopoietic stem cell. The phenotype of primitive human hematopoietic stem cells is controversial, although they have been characterized as small cells which are CD34⁺38⁻, HLA-DR⁻, Thy1⁺/⁻, CD15⁻, Lin⁻, c-kit⁺, 4-hydroperoxy-cyclophosphamide-resistant and rhodamine 123 dull. For the present purpose, a consensus phenotype for human HSC would be CD34⁺38^{-/lo}, Lin⁻c-kit⁺. Equivalent primitive murine stem cells have been characterized as Lin⁻, Sca-1⁺, and c-kit⁺.

In one embodiment, cells which have the phenotype of common lymphoid progenitors (CLP) are used to generate mature T lymphocytes. In mouse, CLPs are defined by the phenotype Lin⁻c-Kit^{lo}IL-7Rα⁺. In humans, CLPs are present in two subsets of Lin⁻CD45RA⁺Thy-1⁻HLA-DR⁺ cells: CD34⁺CD38⁺CD10⁺ and CD34⁺CD38⁻CD7⁺. CD34⁺ cells in particular are generally known as primitive, undifferentiated cells of the embryo, bone marrow, umbilical cord blood and adult tissue, that have the capacity to differentiate.

Examples of T lymphocytes which may be produced include CD4⁺, CD8⁺, and CD4⁺CD8⁺ cells. The T lymphocytes may have αβ or γδ T cell receptors (TCR). They may be naive, activated, or memory T lymphocytes.

The hemapoietic stem cells and lymphoid progenitor cells as well as stromal cells may be cultured on a suspension media or on a three dimensional support by techniques known in the art.

The basic cell medium used in the bioreactor may be any of the widely known media used to support growth and/or differentiation of hemapoietic stem cells and lymphoid progenitor cells and/or stromal cells. For example, the following classical media may be used and supplemented, if desired, with vitamin and amino acid solutions, serum, and/or antibiotics: Fisher's medium (Gibco), Basal Media Eagle (BME), Dulbecco's Modified Eagle Media (D-MEM), Iscoves's Modified Dulbecco's Media, Minimum Essential Media (MEM), McCoy's 5A Media, and RPMI Media.

Specialized media may also be used such as e.g., MyeloCult (Stem Cell Technologies), and Opti-Cell (ICN Biomedicals). If desired, serum free media may be used such as, e.g., StemSpan SFEM (StemCell Technologies), StemPro 34 SFM (Life Technologies) and Marrow-Gro (Quality Biological Inc.).

The culture may be fed at regular intervals with the culture medium. Various other ingredients may be added to the culture media. Such media is herein termed “supplemented”. The media may contain cytokines, extracellular matrices, or other biologically active molecules. The skilled artisan may select the amounts according to the culture system used i.e. size, volume, number and source of cells.

Methods and media for producing antigen specific T cells are also contemplated. Thus, as the hematopoietic stem cells and lymphoid progenitors cells are cultured and undergoing differentiation, they may be immunized with an antigen or antigenic fragment. The T cells produced by the culture which are antigen specific may be identified. T cells may be identified using well known methods in the art such as immunocytochemistry for T cell receptors; for example, using immunocytochemistry for CD4⁺, CD8⁺, αβ or γδ. The antigen used to immunize the culture may be a carbohydrate, peptidoglycan, protein, glycoprotein, virus, tissue mass, cell, cell fragment, or a nucleic acid molecule. The virus, tissue mass, cell, or cell fragment may be live or dead. The antigen may also be a viral antigen or a tumor antigen.

Therapeutic Uses and Treatments

The present invention involves an ex-vivo method of treating subjects that would benefit from having an enhanced number of lymphocytes, specifically T lymphocytes, or for restoring to the subject a depleted population of lymphocytes, specifically T lymphocytes. The method includes the steps of isolating hematopoietic stem cells and/or lymphoid progenitor cells from the subject and expanding the isolated cells in a culture medium including Wnt4 as described herein. The resulting culturing containing the lymphocytes and T cells are then harvested. A therapeutic dose of the harvested cultured cells is then administered back to the subject.

Subjects that would benefit from having an enhanced number of lymphocytes, specifically T lymphocytes, include those that suffered a decrease in lymphocytes as a consequence of disease, radiation or chemotherapy. Mammals which may benefit from an enhancement of lymphopoiesis include those predisposed to, or suffering from, any one or more of the following exemplary conditions: lymphocytopenia; lymphorrhea; lymphostasis; immunodeficiency (e.g., HIV and AIDS); infections (including, for example, opportunistic infections and tuberculosis (TB)); lupus; and other disorders characterized by lymphocyte deficiency.

The ex-vivo method as described herein involves isolating hematopoietic stem and progenitor cells from such sources as the bone marrow, peripheral blood or umbilical cord using methods and materials known in the art. The stem and progenitor cells used in the present method may be enriched, i.e. reduced in number of mature lymphoid cells and cells not of the lymphoid lineage. Methods by which stem and progenitor cells can be isolated and enriched using positive immunoselection are known in the art.

Once the hematopoietic stem and progenitor cells have been cultured as described herein to obtain a population of lymphocytes, a therapeutic dose is administered to a subject. The method of determining an appropriate therapeutic dose is known to those of skill in the art. For example, a therapeutic dose may be 1 to about 2 million cells/kg of the subject's mass. The therapeutic dose may be administered by infusion.

Treatment refers to both therapeutic treatment and prophylactic or preventative measures. Those in need of treatment include those already with the disease or disorder as well as those in which the disease or disorder is to be prevented.

The present invention involves use of the Wnt4 nucleic acid in gene therapy applications, where the Wnt4 gene is expressed at an extra-thymic lymphoid site at a level at least comparable to that in the thymus.

In gene therapy, the gene is introduced into cells in order to achieve in vivo synthesis of a therapeutically effective genetic product, for example for replacement of a defective gene. Gene therapy includes both conventional gene therapy where a lasting effect is achieved by a single treatment, and the administration of gene therapeutic agents, which involves the one time or repeated administration of a therapeutically effective DNA or mRNA.

There are a variety of techniques available for introducing nucleic acids into viable cells. In vivo gene transfer techniques include transfection with viral (typically retroviral) vectors and viral coat protein-liposome mediated transfection. In some situations it is desirable to provide the nucleic acid source with an agent that targets the target cells, such as an antibody specific for a cell surface membrane protein or the target cell, a ligand for a receptor on the target cell, etc. In the present invention, it is desirable to target Wnt4 to sites of the lymphoid tissues.

For therapeutic applications, the Wnt4 polypeptide or cells expressing the Wnt4 polypeptide or the ex-vivo cells containing T cells harvested from culture, may be administered to a mammal, preferably a human, in a physiologically acceptable dosage form. These include intravenous administration as a bolus or by continuous infusion over a period of time or other routes known in the art. The Wnt4 polypeptide also are suitably administered by intratumoral, peritumoral, intralesional, or perilesional routes or to the lymph, to exert local as well as systemic therapeutic effects.

Such dosage forms encompass physiologically acceptable carriers that are inherently non-toxic and non-therapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and PEG. Carriers for topical or gel-based forms of Wnt polypeptides include polysaccharidessuch as sodium carboxymethylcellulose or methylcellulose, polyvinylpyrrolidone, polyacrylates, polyoxyethylene-polyoxypropylene-block polymers, PEG, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The Wnt polypeptide will typically be formulated in such vehicles such that the level at the in vivo active site is at least comparable to that of the thymus.

Therapeutic formulations of Wnt polypeptide are prepared for storage by mixing Wnt polypeptide having the desired degree of purity with optional physiologically acceptable carriers, excipients, or stabilizers (24), in the form of lyophilized cake or aqueous solutions. Acceptable carriers, excipients, or stabilizers are nontoxic to recipients at the dosages and concentrations employed, and include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine, or lysine; monosaccharides, disaccharides, and other carbohydrates including glucose, mannose, or dextrins; chelating agents such as EDTA; sugar alcohols such as mannitol or sorbitol; salt-forming counter-ions such as sodium; and/or non-ionic surfactants such as Tween, Pluronics or polyethylene glycol (PEG).

Wnt4 polypeptide used for in vivo administration must be sterile. This may be accomplished by filtration through sterile filtration membranes, prior to or following lyophilization and reconstitution. Wnt4 polypeptide ordinarily will be stored in lyophilized form or in solution. Therapeutic Wnt4 polypeptide compositions generally are placed into a container having a sterile access port, for example, an intravenous solution bag or vial having a stopper pierceable by a hypodermic injection needle.

For use in the above methods, the invention also provides an article of manufacture or a commercial package or kit, comprising: a container, a label on the container, and a composition comprising Wnt4 as an active agent within the container when used at the indicated level, wherein the composition is effective for supporting proliferation and/or differentiation and/or maintenance of hematopoietic stem/progenitor cells in a mammal. The label on the container indicates that the composition can be used for enhancing proliferation and/or differentiation and/or maintenance of those cells and the active agent in the composition is a Wnt polypeptide. Optionally, the package includes one or more further containers which hold further components in a packaged combination with the container holding the Wnt4 polypeptide. Such optional containers include standard culture media and/or other components for use in the culture and maintenance and/or differentiation of hemapoietic stem and progenitor cells such as IL-7, c-kit ligand and Notch ligand (Notch ligand Delta-like-1).

Experiments and Data Analysis

I. Analysis of Lymphoid Progenitor Populations Shows that Progenitors Committed to T Cell Lineage are Present in LN

To discover the early step in T cell development that occurs in the thymus and the OM-transgenic LN but not the wild-type (wt) LN, we first analyzed populations of lineage-negative (Lin⁻) cells in these organs. We discriminated three subsets of DN1 phenotype cells according to the level of c-Kit expression (negative, low or high) because previous reports showed that this marker identifies cell subsets with different T cell progenitor potential (9, 10, 11, 12).

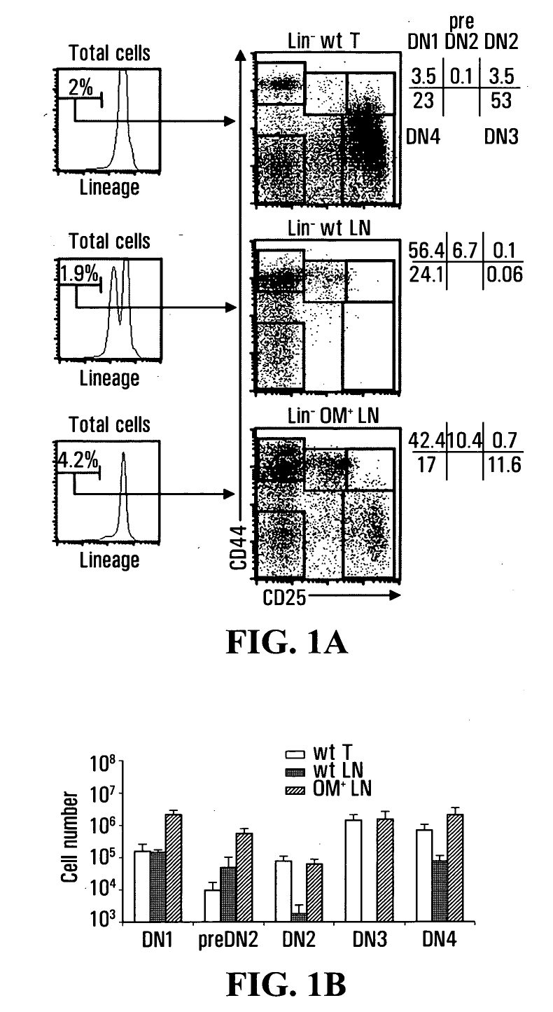

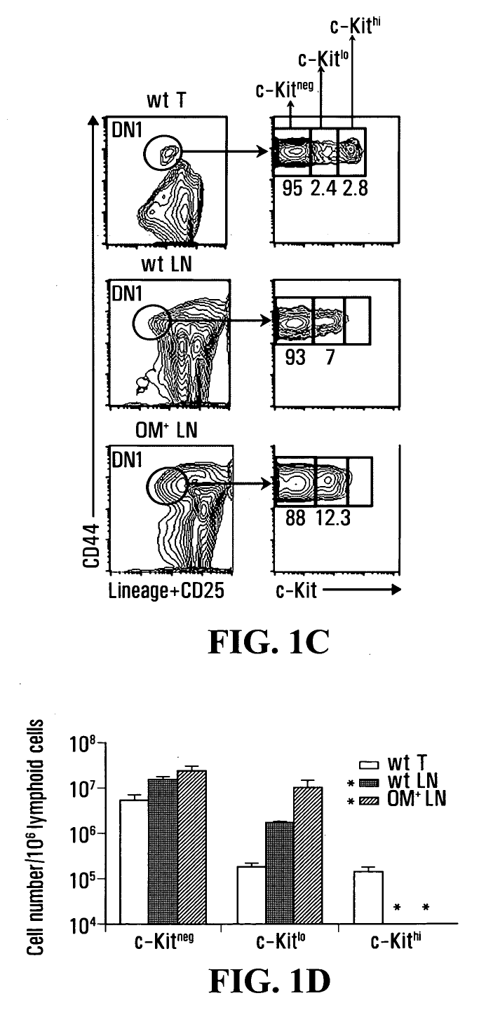

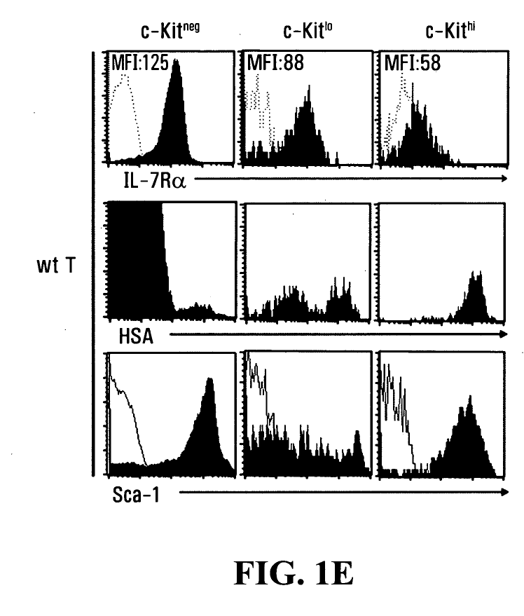

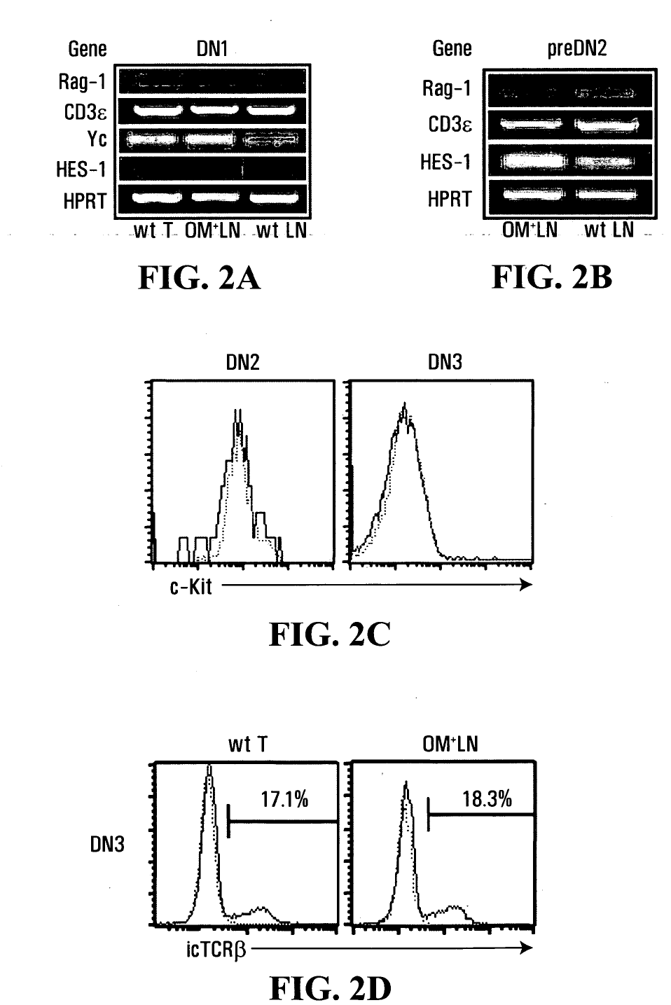

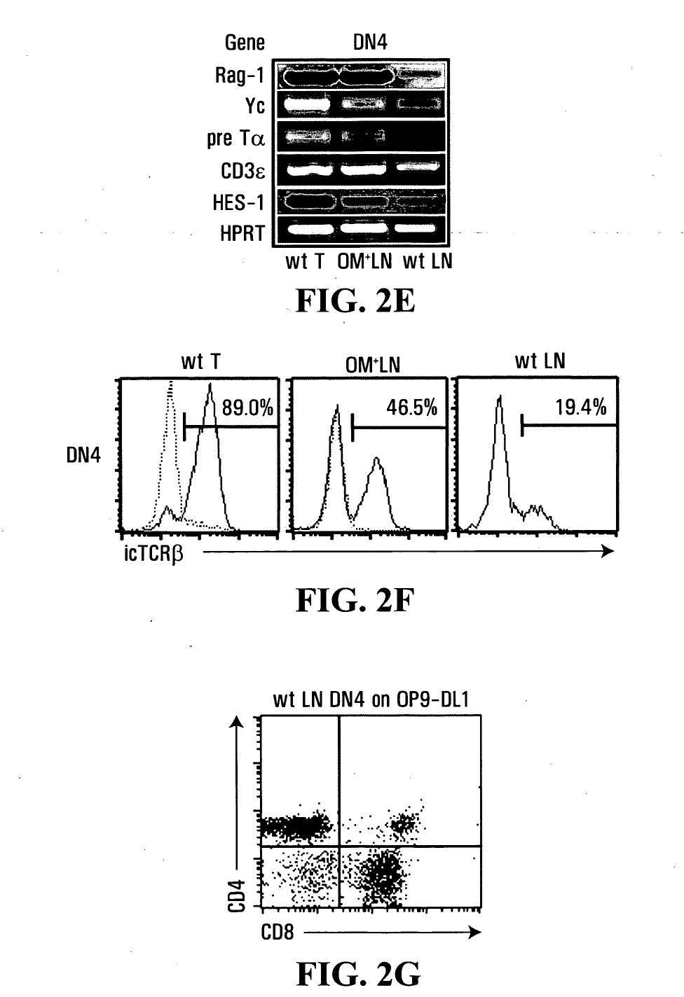

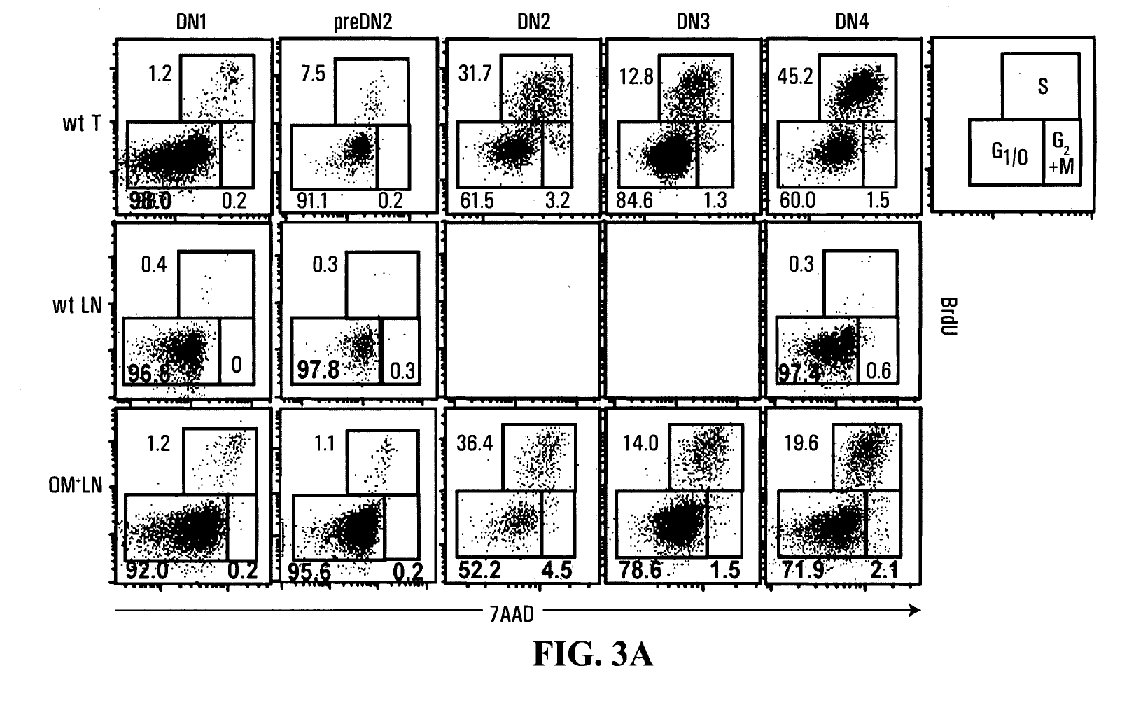

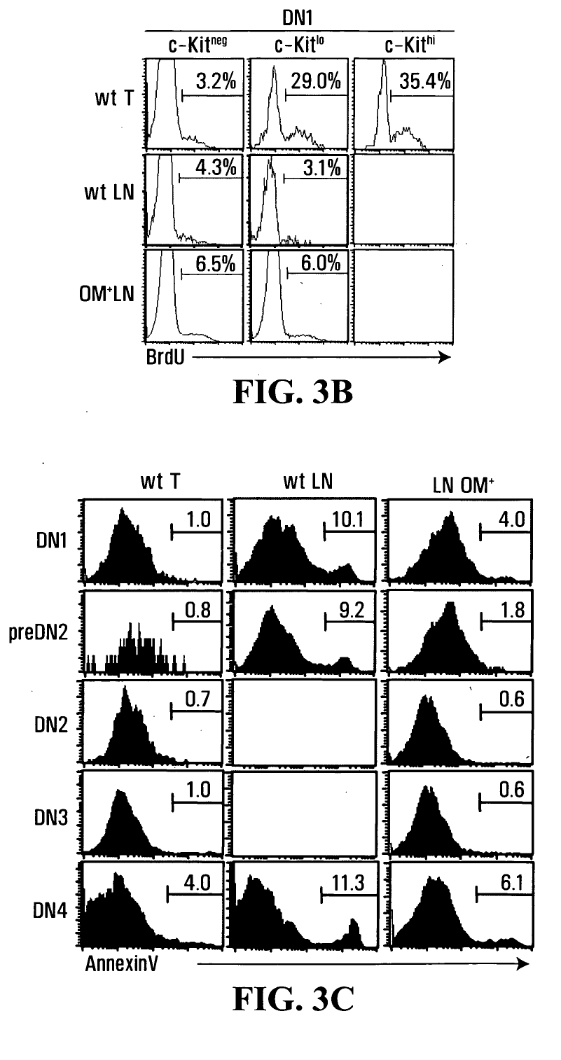

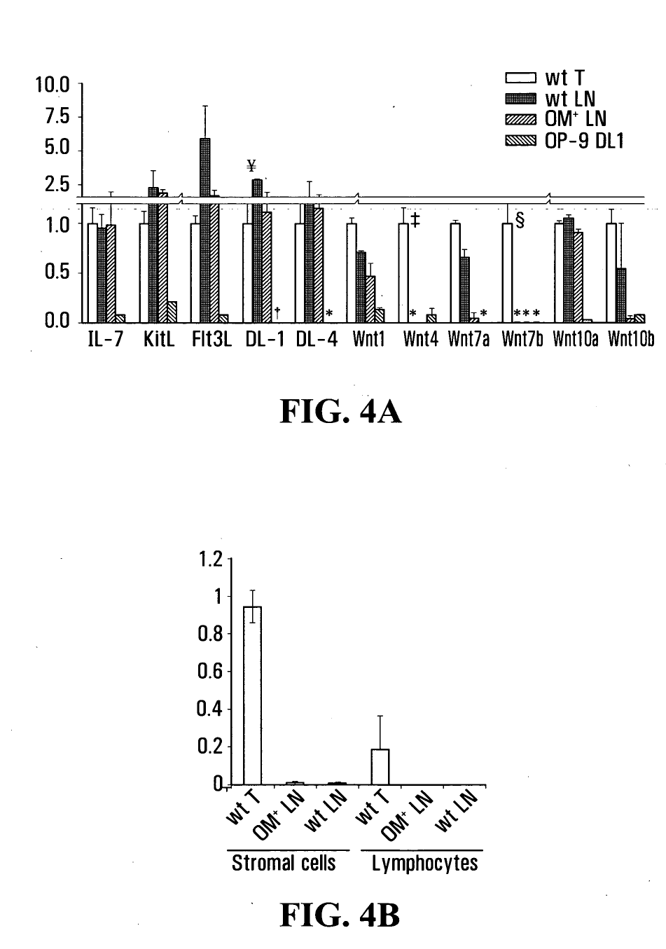

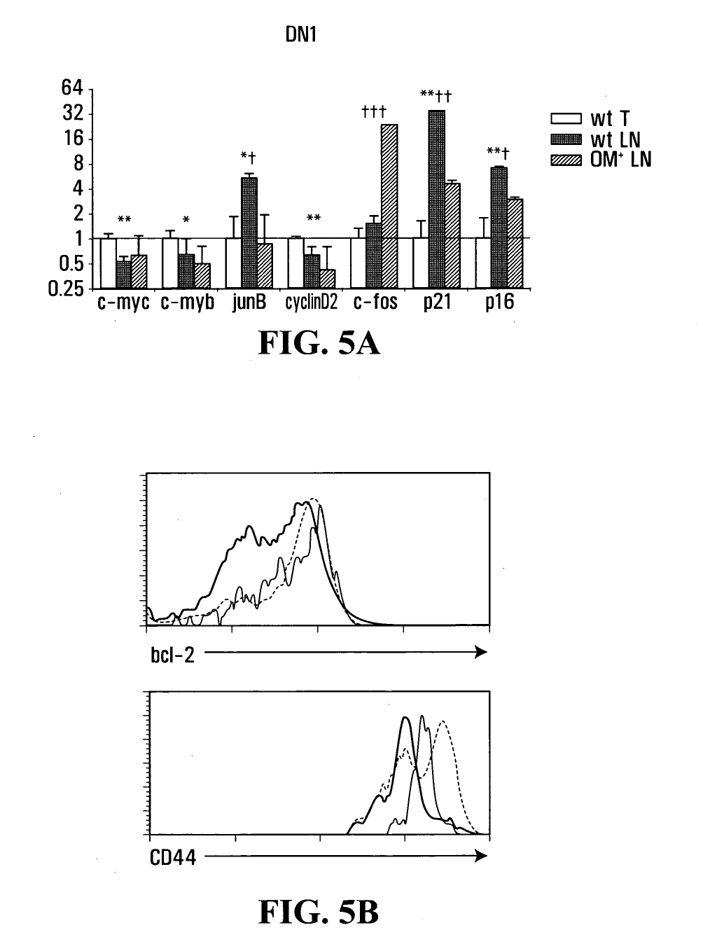

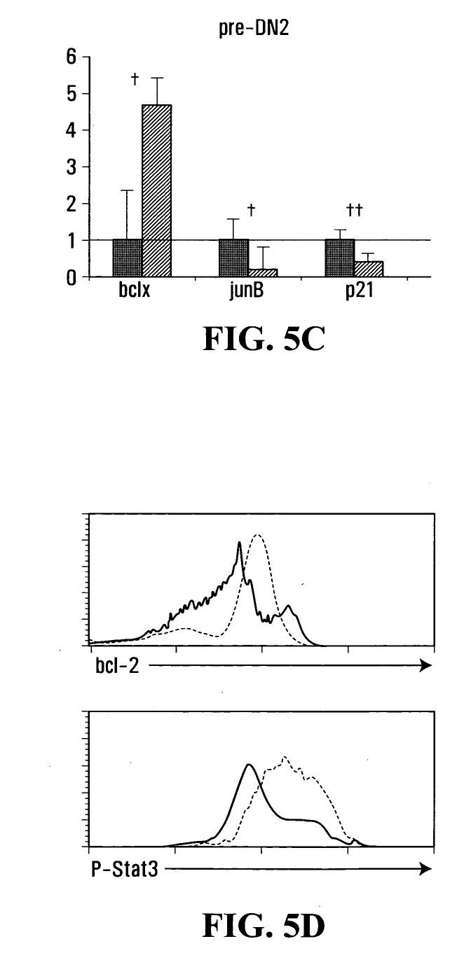

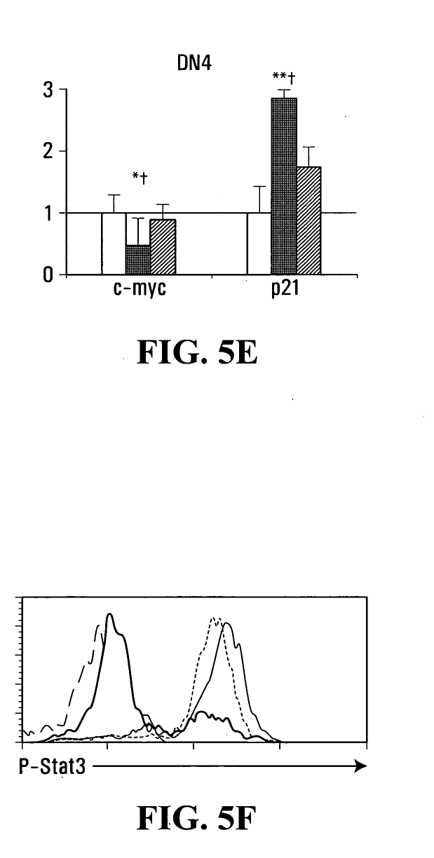

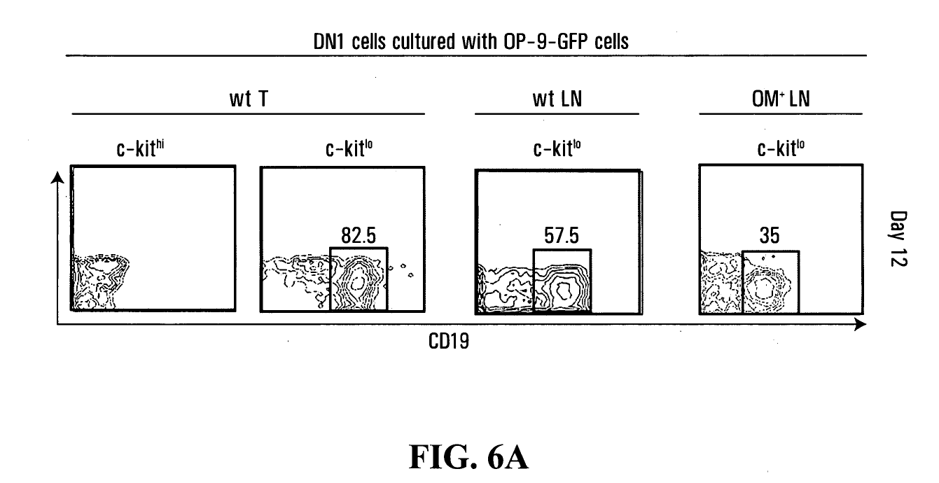

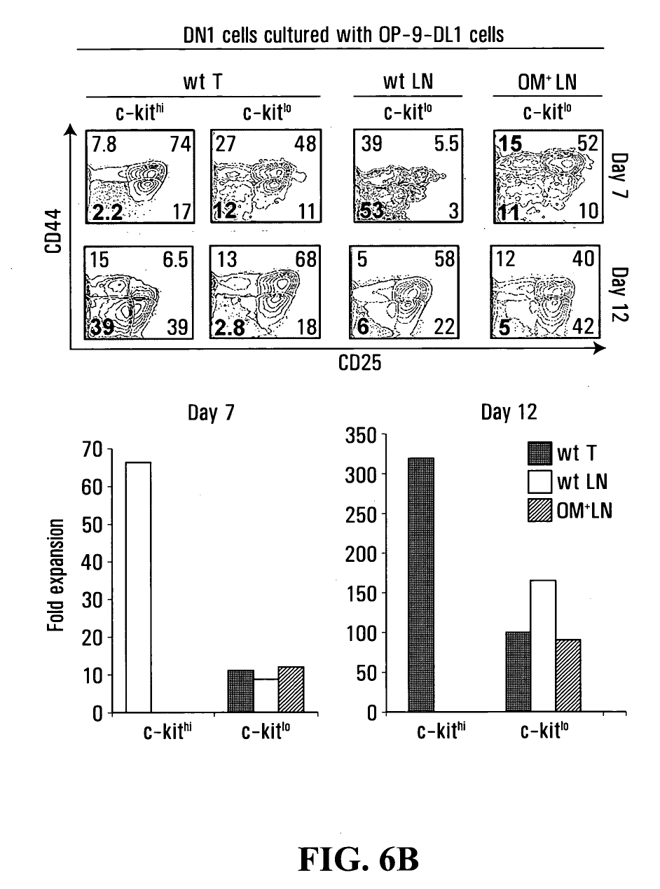

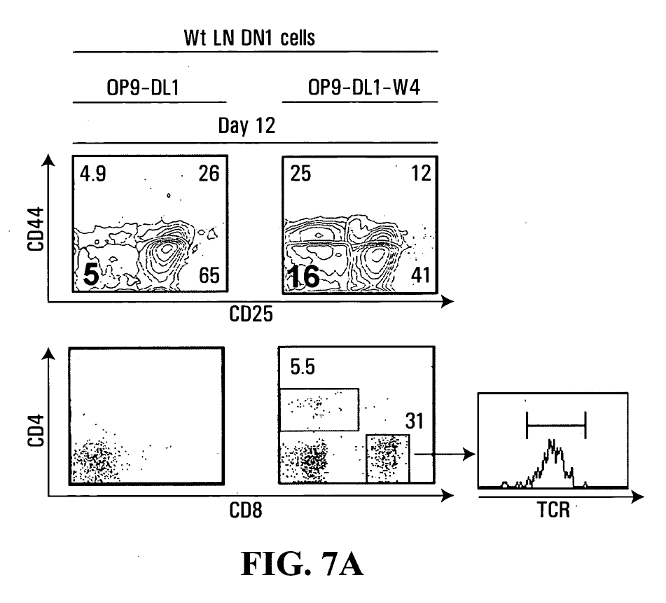

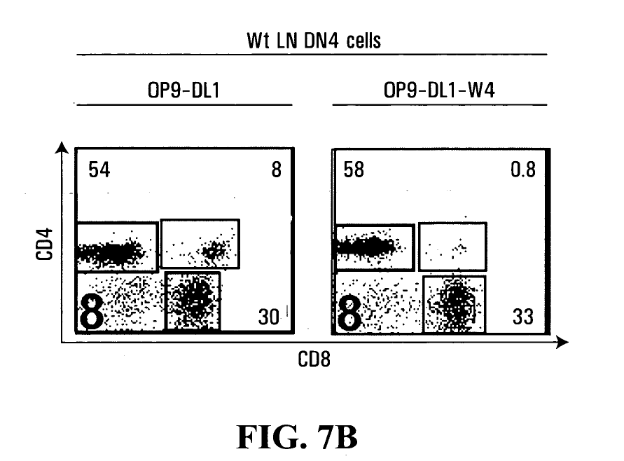

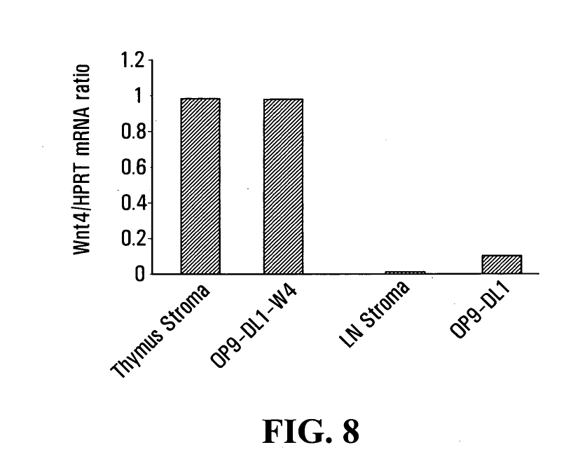

Two types of Lin⁻Sca-1⁺ progenitors can generate T cells in the thymus: c-Kit^{hi}IL-7Rα⁻ and c-Kit^{lo}IL-7Rα⁺. Among thymic DN1 cells, the vast majority of c-Kit⁻ cells were IL-7Rα⁺, CD24(HSA)⁻, and Sca-1⁺, whereas c-Kit^{lo} cells were IL-7Rα⁺CD24^{+/-}Sca-1^{+/-}, and c-Kit⁺ cells were largely IL-7Rα⁻ CD24⁺Sca-1⁺ (FIG. 1c,e). Overall, DN1 phenotype cells were present in similar numbers in the thymus and wt LN and were more abundant in the OM⁺ LN (FIG. 1a,b). However, major discrepancies were found among DN1 cell subsets in the three organs. Strikingly, c-Kit^{hi} DN1 cells were present exclusively in the thymus (FIG. 1c,d). In contrast, c-Kit- and c-Kit^{lo} DN1 phenotype cells were more abundant in the wt LN than the thymus, and even more so in the OM⁺ LN (FIG. 1d). The IL-7Rα, CD24 and Sca-1 phenotype of c-Kit⁻ and c-Kit^{lo} DN1 phenotype cells from the wt and OM⁺ LNs was similar to that of their thymic counterparts (data not shown).

Relative to the thymus, wt and OM⁺ LN showed an increased proportion of cells bearing a pre-DN2 phenotype (CD44⁺CD25^{lo}) (FIG. 1a). T cell commitment requires synthesis of the HES-1 transcription factor and is revealed by the expression of lineage-specific genes. Thus, detection of HES-1, Rag-1 and CD3ε transcripts indicates that DN1 and pre-DN2 subsets in wt and OM⁺ LN contain cells committed to the T lineage (FIG. 2a,b).

II. T Cell Development in LN is Blocked at DN1→DN2 Transition

DN2 and DN3 cells were practically undetectable in wt LN, yet cells with a DN4 phenotype were present (FIG. 1a,b). At the population level, the transcriptome of “illegitimate” wt LN DN4 phenotype cells was not identical to that of genuine thymic DN4 cells, as shown by differences in levels of Rag-1, pre-Tα, and HES-1 transcripts (FIG. 2e). However, at least some of the “illegitimate” DN4 phenotype cells in the wt LN were committed to the T lineage: i) wt LN-derived DN4 cells contained CD3ε transcripts, and about 18% expressed intracytoplasmic TCR β chain (FIG. 2f), ii) when cultured for 7 days in the presence of OP9-DL1 stromal cells, which can support all stages of T cell development, wt LN-derived DN4 cells generated CD4⁺CD8⁺ and single-positive T cells T cells (FIG. 2g).

In contrast to the wt LN, the numbers of cells with DN2, DN3, and DN4 phenotype were similar in OM⁺ LN and thymus (FIG. 1a,b). Furthermore, DN2, DN3, and DN4 cells in the OM⁺ LN were similar to those in the thymus with regard to the levels of several transcripts as well expression of the c-Kit protein (FIG. 2c-f). The sole difference between thymic and OM⁺ LN DN cells was the lower proportion of DN4 cells with rearranged TCR β chain in the the OM⁺ LN (FIG. 2f).

Two major points can be made from these data. First, Lin⁻c-Kit hi IL-7Rα⁻ DN1 cells, whose phenotype corresponds to that of ETPs (9), are present exclusively in the thymus. A corollary is that, at least in the OM⁺ LN, mature T cells can be produced in the absence of c-Kit^{hi}IL-7Rα⁻ DN1 cells. Second, accumulation of pre-DN2 cells in wt and OM⁺ LNs, and emergence of DN2 and DN3 cells in OM⁺ but not wt LN suggest that failure of wt LN to support T cell development is due to a blockade of the the DN1 to DN2 transition that is alleviated in the OM⁺ LN.

III. Proliferation of DN Cells

In the thymus, DN cells proliferate extensively, particularly at the DN2 and DN4 stages. To analyze the proliferation of DN cell subsets in the thymus and LNs, BrdU was injected i.p., mice sacrificed 40 min later, and cell cycle status was determined by staining with anti-BrdU antibody and the fluorescent DNA intercalator 7AAD (13). In addition, the proportion of apoptotic cells was estimated by Annexin V labeling.

As opposed to their thymic counterparts, all DN phenotype cells in the wt LN were arrested at the G1 phase of the cell cycle with virtually no cells in S phase (FIG. 3a). In the OM⁺ LN, the percentage of cells in S phase was similar to thymocytes for DN1, DN2, and DN3 cells, but significantly lower for the pre-DN2 and DN4 subsets (FIG. 3a). Among DN cells in the wt LN, lack of proliferation was correlated with higher proportion of apoptotic cells compared to the thymus and the OM⁺ LN (FIG. 3c).

Since DN1 cells found in lymphoid organs are heterogeneous (FIG. 1c), we sought to provide a more accurate estimation of their mitotic behavior by assessing BrdU incorporation in cell subsets expressing different levels of c-Kit (FIG. 3b). In the thymus, BrdU⁺ DN1 cells were found mainly in the c-Kit^{lo} and c-Kit^{hi} cell subsets (FIG. 3b). In contrast, BrdU incorporation by DN1 cells in LNs was independent of c-Kit level, being of similar and relatively modest magnitude among c-Kit^{neg} and c-Kit^{lo} cells, and increased about two-fold in OM⁺ relative to wt LN (FIG. 3b). Thus, cell cycle status of DN1 cells was correlated with c-Kit expression in the thymus but not wt or OM⁺ LN. The low level of BrdU incorporation among c-Kit^{lo} DN1 cells in the LNs relative to the thymus suggests that the LN stroma fails to provide either c-Kit ligand or another signal that promotes proliferation of c-Kit^{lo} DN1 cells in the thymus.

In the absence of DN2 and DN3 cells, the presence in the wt LN of DN4 phenotype cells displaying evidence of T lineage commitment (FIG. 1a, 2e-g) could be due to extensive proliferation of rare (undetectable) DN3 cells that escaped blockade at the pre-DN2 stage. The practical absence of cycling DN4 cells in the wt LN (FIG. 3a) argues against this and rather suggests that a cryptic pathway generates illegitimate DN4 cells directly from DN1 cells.

IV. Key Differences Between Thymus and in Stroma Involve Wnt Proteins, Specifically Wnt4

The above data show that failure of wt LN to support T cell development is due to inability to complete the DN1 to DN2 transition. This defect is largely alleviated in OM⁺ LN. T cell development is however not entirely thymus-like in the OM⁺ LN where accumulation of pre-DN2 cells and relatively low proliferation of DN4 phenotype cells were found. Signals required for the development of thymocytes at the DN1-DN2 stage are initiated by key ligands that control proliferation and survival (IL-7, kit ligand, and Wnt proteins), cell adhesion (Wnt), and T cell lineage commitment (Delta-like Notch-1 ligands). Expansion of the DN4 cell subset requires expression of the pre-TCR (at the DN3 stage), which has no ligand, and Wnt signals.

We therefore performed quantitative PCR on the stroma of lymphoid organs to evaluate the expression profile of IL7, Kit ligand, Delta-like proteins, and 6 Wnt proteins which are normally present in the thymuS (14, 15). We also assessed expression of the fms-like tyrosine kinase-3 (flt3) cytokine gene, because, although it is not essential for T cell development, it may influence the survival of lymphoid progenitors (16).

We found no deficit of the following transcripts in the wt LN relative to the thymus: IL-7, Kit ligand, flt3, Delta-like-1 and -4, Wnt1, Wnt7a, Wnt10a and Wnt10b (FIG. 4a). Furthermore, none of these transcripts was more abundant in the OM⁺ compared to wt LN (FIG. 4a). However, two salient differences were observed between the thymus and LNs: Wnt4 and Wnt7b transcripts were present in the thymus but absent in the LN (P<0.0001 and P<0.005, respectively).

Though we cannot formally exclude that lack of Wnt7b in the LN may be biologically relevant, we elected to focus our attention (and culture experiments described below) on Wnt4 for the following reasons:
i) Wnt4, which regulates FoxN1 expression, is the most abundantly expressed Wnt family member in both embryonic thymic epithelium as well as mature thymic cortical epithelium (14, 15);
ii) OP9-DL1 stromal cells which can support all steps of T cell development express Wnt4 but not Wnt7b (FIG. 4a).

Since stromal fractions may be contaminated by a few adherent lymphoid cells, we evaluated Wnt4 transcripts in thymus lymphoid and stromal fractions by quantitative RT-PCR and confirmed that stromal cells were the main if not the sole site of Wnt4 transcription in the thymus (FIG. 4b).

V. Wnt and LIF/OM Signaling Pathways in DN Phenotype Cells

To evaluate whether and how lack of Wnt4 could hamper T cell development, we studied by quantitative PCR the expression of genes that have been implicated in thymocyte development and shown to be regulated by Wnt signals in various cell types. We performed these studies in the two subsets of DN phenotype cells that are present in significant numbers in both the thymus and wt LN, that is, DN1 and DN4 cells (FIG. 1a,b).

In line with what is known about Wnt signaling, transcript levels of c-myb, c-myc, and cyclin D2 were lower while those of junB, p16^{INK4a} and p21^{Cip1/WAF1} were higher in wt LN compared to thymus DN cells (FIG. 5a,e). However, c-fos levels were not deficient in the wt LN relative to thymus DN1 cells (FIG. 5a). Thus, aside from c-fos levels, transcript profiles point to a dearth of Wnt signals in DN cells from the wt LN relative to the thymus. This suggests that in DN cells, Wnt4 signaling has a non-redundant effect on genes such as c-myb, c-myc, and junB, but is not essential for induction of c-fos.

Bovine OM binds only to the LIF receptor in mouse. Whilst extrathymic T cell development in mice expressing OM under the Lck promoter (LckOM) must therefore be induced by OM binding to the LIF receptor, whether this interaction occurs specifically in immature T cells has not been determined. To address this, we studied the three subsets of DN phenotype cells present in both the wt and OM⁺ LNs (DN1, pre-DN2, and DN4; cf. FIG. 1a,b).

Signals from the LIF receptor partially overlap with those induced by Wnt signaling and have a similar impact on transcription of c-fos, junB, p16^{INK4a}, p21^{Cip1/WAF1} and c-myc. Comparison of transcript levels in the OM⁺ relative to wt LN supports the idea that OM signals in DN cells from the OM⁺ LN compensate for the lack of Wnt signalling: levels of c-fos and c-myc were higher while those of junB, p16^{INK4a}, and p21^{Cip1/WAF1} were decreased in DN cells from the OM⁺ relative to the wt LN (FIG. 5a,c,e). Supplementary evidence for OM signaling in DN cells from the OM⁺ LN included upregulation of CD44 in DN1 cells (FIG. 5a), of bcl-2 in DN1 and pre-DN2 cells (FIG. 5b,d), of bcl-x_{L} in pre-DN2 cells (FIG. 5c), and of phosphoSTAT3 in pre-DN2 and DN4 cells (FIG. 5d,f).

VI. In Vitro Differention of c-Kit^{lo} and c-Kit^{hi} Progenitors

We next asked whether culture with stromal cells expressing Wnt4 could allow DN1 phenotype cells from the LNs to undergo T lineage differentiation. OP9-DL1 express Wnt4, albeit at lower levels than thymic stromal cells (FIG. 4). Thus, we cultured the following subsets of DN1 phenotype cells in the presence of OP9-DL1 stromal cells: c-Kit^{hi} (IL-7Rα⁻Sca-1⁺) cells from the thymus, as well as c-Kit⁻ (IL-7Rα⁺) and c-Kit^{lo} (IL-7Rα⁺) from the thymus, wt LN, and OM⁺ LN. Sca-1⁺ is uniformly expressed on c-Kit^{hi} cells but is present only on about 80% and 40% of c-Kit⁻ and c-Kit^{lo} DN1 cells, respectively (FIG. 1e). Thus, for c-Kit⁻ and c-Kit^{lo} cells, Sca-1⁺ and Sca-1⁻ subsets were sorted and analyzed separately.

As expected, for all cell subsets tested no development toward the T lineage was observed in the presence of OP9 cells, that is, in the absence of the Notch ligand Delta-like 1. In the presence of OP9-DL1 cells, T cell differentiation was observed with thymic c-Kit^{hi} cells, and Sca-1⁺c-Kit^{lo} cells from the three lymphoid organs (FIG. 6b) In contrast, no T cell differentiation (appearance of DN2 phenotype cells) was observed with c-Kit⁻Sca-1⁻, c-Kit⁻Sca-1⁺, and c-Kit^{lo}Sca-1⁻ subsets. The behavior in culture of c-Kit⁻ and c-Kit^{lo} cell subsets was not influenced by their site of origin (thymus, wt LN or OM⁺ LN) (FIG. 6).

c-Kit^{hi} (thymic) DN1 cells cultured with OP9-DL1 cells proliferated extensively, generated DN4 cells after 12 days (FIG. 6b) and CD4⁺CD8⁺ as well as single positive T cells after 18 days (data not shown). In comparison to c-Kit^{hi} DN1 cells, Sca-1⁺c-Kit^{lo} DN1 cells (from the thymus or LN) showed two deficits: i) in terms of absolute numbers, they accumulated to lower levels on day 7 and 12, and ii) their progeny showed a very low proportion of DN4 cells on day 12 (FIG. 6b). Furthermore, Sca-1⁺c-Kit^{lo} differed from c-Kit^{hi} DN1 thymic cells in that only the former generated substantial numbers of CD19⁺ B cells when cultured on OP9 cells (FIG. 6a). Thus, when cultured with (Wnt4^{lo}) OP9-DL1 cells, Sca-1⁺c-Kit^{lo} DN1 cells from the thymus and LN progress well up to the DN3 stage, but expansion of their DN4 cell progeny is limited.

VII. Thymus-Like Level of Wnt Promotes Differentiation of Lymphoid Progenitors into Mature T Cells

OP9-DL1 stromal cells express only low levels of Wnt4, about 15% those of the thymus stroma (FIG. 4). We therefore engineered OP9-DL1 cells to express levels of Wnt4 transcripts similar to the thymus using a construct in which Wnt4 is expressed under control of the CMV promoter. We then tested their ability to support the development of Sca-1⁺c-Kit^{lo} LN DN1 phenotype cells.

We determined the expression levels of Wnt4 by real-time PCR (FIG. 8). This method relies on the detection and quantification of a fluorescent reporter, whose signal increases proportionately to the amount of gene-specific cDNA in a reaction. The real-time PCR equipment (in this case by Applied Biosystems) monitors and compiles fluorescent signals emitted during the reaction, indicating amplicon production during each amplification cycle. By assessing fluorescence emission at each cycle, PCR amplification is evaluated during the exponential phase, which correlates with the initial amount of target gene template.

A standard curve is generated for an endogenous house-keeping gene (in this case, the HPRT gene) as well as for each specific target gene using sequential dilution of a reference sample (in this case, generated from RNA isolated from thymic stromal cells). Real-time PCR is performed simultaneously on both the reference sample and the experimental samples. As shown in FIG. 8, these were comprised of samples generated from RNA extraction of OP9-DL1 cells, OP9-DL1-Wnt4 cells, stromal cells from wt LN, and stromal cells from OM+ LN.

A relative value for target gene expression in each sample is extrapolated from the standard curve generated from the reference sample. For each sample, the ratio of target gene/HPRT expression is calculated. The reference sample ratio (thymic stroma) is then arbitrarily set at 1 and each sample ratio values are then transformed proportionately.

Provision of thymus-like amounts of Wnt4 by OP9-DL1-W4 stromal cells increased by three-fold the number of DN4 cells generated from Sca-1⁺ c-Kit^{lo} LN DN1 cells on day 12 (FIG. 7a). Moreover, overexpression of Wnt4 on stromal cells allowed Sca-1⁺c-Kit^{lo} LN DN1 cells to generate TCRαβ single-positive T cells as early as on day 12 of culture (FIG. 7a).

In addition to enhancement of DN4 cells expansion, Wnt4 may regulate differentiation events downstream of the DN4 stage since it induced a modest but reproducible shortening of the time required for transition from the CD4⁺CD8⁺ to single-positive phenotype (FIG. 7b). Thus, increased expression of Wnt4 by Wnt4-overexpressing OP9-DL1 cells was sufficient to allow LN Sca-1⁺c-Kit^{lo} cells to generate mature T cells.

We found that we can induce T-cell generation from human peripheral blood CD34⁺ cells. We plated at least 10,000 CD34⁺ cells per well and cultured them on OP9-DL1 stromal cells in the presence of IL-7 and FLT3L for 30-45 days. We think the yield may be increased by plating the CD34⁺ cells on OP9-DL1-Wnt4 stromal cells.

VIII. Use of Wnt4 to Induce Extrathymic T-Cell Development In Vivo

Mouse Wnt4 gene (sequence accession number NM_{—}009523) was inserted into the multiple cloning site of pMSCV-IRES-GFP such that expression of Wnt4 was linked to GFP expression via the internal ribosome entry site (IRES). Expression of Wnt4 can thus be monitored by monitoring GFP expression due to co-expression of the two genes.

Transfection in amphotropic vesicular stomatitis virus (VSV) pseudotyped retrovirus packaging cells 293GPG is performed using Lipofectamine 2000, according to the manufacturer's instructions. The retroviral titers of the pMSCV-IRES-GFP control virus and the pMSCV-Wnt4-IRES-GFP is assessed by transfer of GFP expression to NIH-3T3 cells. This is estimated indirectly by transducing a known number of NIH-3T3 cells with different volumes of retroviral containing supernatant. Then, increasing volumes of supernatant (1, 0.1, 0.01 mL) are plotted against percentage of fluorescent target cells. Transduced cells are analyzed after 48 hours by flow cytometry. The titer is calculated from the volumes corresponding to the linear slope of the curve according to the following formula:

$$Viral Titer : \frac{NIH - 3 T3 cell no . \times \% of fluorescent cells}{Volume of viral Supernatant ( mL )}$$

High-titer virus-containing supernatants are used to infect a packaging cell line, such as the ecotropic packaging cell line GP+E-86 kindly provided by G. Sauvageau in this case. Integrity of protein and protein production is assessed on day 6 post-transduction of GP+E-86 cells by western blot assays on protein extracts from NIH-3T3 and GP+E-86 transduced cells using a mouse anti-Wnt4 antibody. Confirmation of provirus integrity is achieved by Southern blot on day 7 post-transduction.

For assessment of in vivo Wnt4 over-expression within the lymphoid compartments of mammals, primary mouse bone marrow cells are transduced with Wnt4-expressing virus. Four days following intravenous injection of 150 mg/kg of 5-fluorouracil to 6-10 month old C57BL/6 mice, bone marrow cells are harvested and cultured for 48 hours in Dulbecco's modified eagle's medium (DMEM) supplemented with 15% foetal bovine serum (FBS), 10 ng/mL hIL-6, 6 ng/mL mIL-3, and 100 ng/mL mSF. Treated bone marrow cells are then cocultured with above mentioned irradiated (1500 cGy x-ray) GP+E-86 viral producer cells, producing control or Wnt4+ murine moloney leukemia virus, (MuMoLV). This proceeds for 48 hours in the same medium with the addition of 5 ug/mL protamine sulfate. Loosely adherent and nonadherent cells are recovered from the cocultures and incubated an additional 48 hours in the same medium without protamine sulfate. Retrovirally transduced bone marrow cells are then selected based on GFP expression using fluorescence-activated cell sorter.

Irradiated murine recipients (1200 cGy) receive intravenous injection of a suspension of either 100% GFP+ bone marrow cells or a mixture of 1:1 of GFP+:GFP− cells (300 000 cells injected/mice). At different time-points (15, 30, 45, 60 and 90 days post-injection) bone marrow, thymus, lymph nodes, spleen and blood of infected mice are harvested. Assessment of T cell development is performed by flow cytometry with antibodies directed against CD44, CD25, c-kit, Sca-1 and a cocktail of lineage markers (CD3, B220, Ter119, Gr-1 and Ly6C).

IX. Materials and Technical Protocols

Mice. C57BL/6J (B6) mice were purchased from The Jackson laboratory (Bar Harbor, Me.). OM-transgenic mice on a C57BL/6J background have been previously described (17, 18).

Flow cytometry analysis and cell sorting. The following antibodies were used: biotin and PE-Cy7 anti-CD8α (53-6.7), biotin anti-CD8β (53-5.8), APC-Cy7 anti-CD4, biotin anti-NK1.1 (PK136), biotin, APC and FITC anti-TCRβ (H57), biotin anti-TCRγδ (GL-3), FITC and PE anti-CD44 (IM7), biotin, APC-Cy7, PE and APC anti-CD25 (PC61), biotin mouse lineage panel [CD3ε, CD11b, CD45R/B220, Ly6C, Ly6G (GR-1), TER-119/erythroid cells (Ly-76)], purified anti-CD127 (IL-7Rα, A7R34) detected with Goat anti-rat FITC, APC anti-CD117 (c-Kit, 2B8), FITC anti-CD24 (HSA), Pe-Cy5 and PE anti-Sca-1 (E13-161.7), FITC anti-BrdU (3D4) with its isotype control (MOPC-21), FITC anti-Bcl-2 (3F11) with its isotype control (A19-3), FITC anti Annexin. All biotinylated antibodies were detected with streptavidin percp or PE-Cy7. Anti-CD127 was purchased from eBioscience (San Diego, Calif.) and other antibodies mentioned above were purchased from BD Pharmingen (San Diego, Calif.) and Cedarlane Laboratories (Hornby, ON Canada). Polyclonal purified anti Phosoho-STAT3 (Tyr705) (Signalling Technology; Beverly, Mass.) was detected with FITC Goat anti-rabbit IgG F(ab)2 (Abcam; Cambridge, Mass.). Cells were analyzed on a FACSCalibur flow cytometer using CellQuest software and sorted on a six laser FACSDiVa (BD Biosciences, Mountain View, Calif.). Intracellular staining was done as previously described for BrdU (19), TCRβ and Bcl-2 (20), and Phospho-Stat3 (21).

Semi-quantitative RT-PCR. RNA was prepared from cells sorted in trizol reagent (Invitrogen; Burlington, Ontario, Canada) followed by chloroform extraction and RNA precipitation following the manufacturer's instructions. We performed RT-PCR with Quiagen One Step RT-PCR Kit using previously described RT-PCR conditions and primers (Hprt, Rag-1, γ_{c}, preTα, CD3ε, HES-1) (22).

Quantitative RT-PCR. Real-Time RT-PCR was performed with an ABI Prism Sequence Detection System 7700 (Applied Biosystems; Foster City, Calif.), using TaqMan One-Step RT-PCR Master Mix Reagents Kit for lymphoid progenitors and TaqMan Universal PCR Master Mix for stromal cells (Applied Biosystems). mRNA from stromal cells was extracted in trizol reagent and reverse transcription was carried out using SuperScript II RNaseH Reverse Transcriptase (Invitrogen, Burlington, Ontario, Canada). Triplicate wells were averaged and the target gene values were normalized for Hprt content. We used specific primers and probes (TaqMan gene expression assays) from Applied Biosystems.

T cell Progenitors and OP9 Cell Cocultures. T cell progenitors were isolated from thymus and lymph nodes of 6 to 8 week wt and OM⁺ mice. DN1 and DN4 Lin⁻ progenitors were sorted according to surface expression of CD44, c-kit and Sca-1. Sorted cells were seeded at 4×10⁴ cells/well, unless stated otherwise, onto 24 or 6 well tissue plates containing a confluent monolayer of OP9 cells expressing GFP alone, DL-1 or DL-1 and Wnt4 (obtained from Dr. J. C. Zuniga-Pflucker, University of Toronto). Wnt4 plasmid (Wnt4 cDNA under CMV promoter in pUSEamp(+), the complete sequence of which is available from the supplier Upstate biotechnology, Lake Placid, N.Y.) transfection of OP-9 DL-1 cells was carried out using FuGene 6 (Roche Biochemicals, Rotkreuz, Switzerland) according to manufacturer's instructions. All co-cultures were performed in the presence of IL-7 and Flt3L (Peprotech, Rocky Hill, N.J.)(23). Cocultures were harvested by forceful pipetting at the indicated time points and stained for flow cytometry analysis.

### REFERENCE LIST

1. Schmitt, T. M. & Zuniga-Pflücker, J. C. Induction of T cell development from hematopoietic progenitor cells by Delta-like-1 in vitro. Immunity 17, 749-756 (2002).
2. Schmitt, T. M. et al. Induction of T cell development and establishment of T cell competence from embryonic stem cells differentiated in vitro. Nat. Immunol. 5, 410-417 (2004).
3. Guy-Grand, D. et al. Extrathymic T cell lymphopoiesis: Ontogeny and contribution to gut intraepithelial lymphocytes in athymic and euthymic mice. J. Exp. Med. 197, 333-341 (2003).
4. Boileau, C., Houde, M., Dulude, G., Clegg, C. H., & Perreault, C. Regulation of extrathymic T cell development and turnover by Oncostatin M. J. Immunol. 164, 5713-5720 (2000).
5. Antica, M. & Scollay, R. Development of T lymphocytes at extrathymic sites. J. Immunol. 163, 206-211 (1999).
6. Nelson, W. J. & Nusse, R. Convergence of Wnt, β-catenin, and cadherin pathways. Science 303, 1483-1487 (2004).
7. Staal, F. J. & Clevers, H. C. Wnt signaling in the thymus. Curr. Opin. Immunol. 15, 204-208 (2003).
8. Arber, C. et al. Common lymphoid progenitors rapidly engraft and protect against lethal murine cytomegalovirus infection after hematopoietic stem cell transplantation. Blood 102, 421-428 (2003).
9. Allman, D. et al. Thymopoiesis independent of common lymphoid progenitors. Nat. Immunol. 4, 168-174 (2003).
10. Martin, C. H. et al. Efficient thymic immigration of B220⁺ lymphoid-restricted bone marrow cells with T precursor potential. Nat. Immunol. 4, 866-873 (2003).
11. Porritt, H. E. et al. Heterogeneity among DN1 prothymocytes reveals multiple progenitors with different capacities to generate T cell and non-T cell lineages. Immunity 20, 735-745 (2004).
12. Ceredig, R. & Rolink, T. A positive look at double-negative thymocytes. Nat. Rev. Immunol. 2, 888-897 (2002).
13. Chi, T. H. et al. Sequential roles of Brg, the ATPase subunit of BAF chromatin remodeling complexes, in thymocyte development. Immunity. 19, 169-182 (2003).
14. Balciunaite, G. et al. Wnt glycoproteins regulate the expression of FoxN1, the gene defective in nude mice. Nat. Immunol. 3, 1102-1108 (2002).
15. Pongracz, J., Hare, K., Harman, B., Anderson, G., & Jenkinson, E. J. Thymic epithelial cells provide Wnt signals to developing thymocytes. Eur. J. Immunol. 33, 1949-1956 (2003).
16. Sitnicka, E. et al. Key role of flt3 ligand in regulation of the common lymphoid progenitor but not in maintenance of the hematopoietic stem cell pool. Immunity 17, 463 (2002).
17. Clegg, C. H., Rulffes, J. T., Wallace, P. M., & Haugen, H. S. Regulation of an extrathymic T-cell development pathway by oncostatin M. Nature 384, 261-263 (1996).
18. Boileau, C., Houde, M., Dulude, G., Clegg, C. H., & Perreault, C. Regulation of extrathymic T cell development and turnover by Oncostatin M. J. Immunol. 164, 5713-5720 (2000).
19. Penit, C., Lucas, B., & Vasseur, F. Cell expansion and growth arrest phases during the transition from precursor (CD4⁻8⁻) to immature (CD4⁺8⁺) thymocytes in normal and genetically modified mice. J. Immunol. 154, 5103-5113 (1995).
20. Labrecque, N. et al. How much TCR does a T cell need? Immunity 15, 71-82 (2001).
21. Cavallo, F. et al. Interleukin 12-activated lymphocytes influence tumor genetic programs. Cancer Res. 61, 3518-3523 (2001).
22. Waskow, C., Paul, S., Haller, C., Gassmann, M., & Rodewald, H. R. Viable c-Kit^{w/w} mutants reveal pivotal role for c-Kit in the maintenance of lymphopoiesis. Immunity 17, 277-288 (2002).
23. Schmitt, T. M. & Zuniga-Pflücker, J. C. Induction of T cell development from hematopoietic progenitor cells by Delta-like-1 in vitro. Immunity 17, 749-756 (2002).
24. Remington's Pharmaceutical Sciences, 16th edition, Osol, A., Ed., (1980).
25. Austin et al. Blood. 1997. 89(10):3624-3635.
26. Xiao et al. 2003. J. Biol. Sci. 278(32):29954-29962.
27. Sheldahl et al. 2003. J. Cell Bio. 161(4):769-777.

1. Media for supporting lymphopoiesis in culture, the media comprising Wnt4 protein at a level which is at least equivalent to that found naturally in the thymus.

2. The media of claim 1 wherein the Wnt4 protein is present on the surface of a Wnt4-expressor cell.

3. A culture comprising lymphoid progenitor cells and/or T lymphocytes in the media defined in claim 1.

4. A culture comprising lymphoid progenitor cells and/or T lymphocytes in the media defined in claim 2.

5. A culture comprising Wnt4-expressor cells, and lymphoid progenitor cells, and/or T lymphocytes, wherein the culture comprises Wnt4 protein at a level which is at least equivalent to that found naturally in the thymus.

6. A method for obtaining T lymphocytes from lymphoid progenitor cells, the method comprising the step of providing Wnt4 protein to the lymphoid progenitor cells in culture at a level which is at least equivalent to that found naturally in the thymus.

7. The method of claim 6 wherein the Wnt4 protein is provided by a Wnt4-expressor cell which is present in the culture.

8. The media of claim 1 wherein the level of Wnt4 protein is at least 100 ng/mL of media.

9. The culture of claim 3 wherein the level of Wnt4 protein is at least 100 ng/mL of media.

10. The culture of claim 5 wherein the level of Wnt4 protein is at least 100 ng/mL of media.

11. The method of claim 6 wherein the level of Wnt4 protein is at least 100 ng/mL of media.

12. The method of claim 7 wherein the level of Wnt4 protein is at least 100 ng/mL of media.


SEQUENCE LISTING (2 sequences)

SEQ ID NO: 1 - protein, 351 aa, mus musculus
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDIRSGVLGTRGRTCNKTSKAIDGCELLCCGRGFHTAQVELAERCGCRFHWCCFVKCRQCQRLVEMHTCR

SEQ ID NO: 2 - protein, 351 aa, homo sapiens
MSPRSCLRSLRLLVFAVFSAAASNWLYLAKLSSVGSISEEETCEKLKGLIQRQVQMCKRNLEVMDSVRRGAQLAIEECQYQFRNRRWNCSTLDSLPVFGKVVTQGTREAAFVYAISSAGVAFAVTRACSSGELEKCGCDRTVHGVSPQGFQWSGCSDNIAYGVAFSQSFVDVRERSKGASSSRALMNLHNNEAGRKAILTHMRVECKCHGVSGSCEVKTCWRAVPPFRQVGHALKEKFDGATEVEPRRVGSSRALVPRNAQFKPHTDEDLVYLEPSPDFCEQDMRSGVLGTRGRTCNKTSKAIDGCELLCCGRGFHTAQVELAERCSCKFHWCCFVKCRQCQRLVELHTCR